(19) United States
(12) Patent Application Publication
(10) Pub. No.: US 2012/0077795 A1
(43) Pub. Date: Mar. 29, 2012

(54) BIS-PYRIDYLPYRIDONES AS MELANIN-CONCENTRATING HORMONE RECEPTOR 1 ANTAGONISTS

(75) Inventors: Siegfried Benjamin Christensen IV, King of Prussia, PA (US); Donghui Qin, King of Prussia, PA (US); Shi Lu, Shanghai (CN); Shuai Wei, Shanghai (CN); Nianyong Yang, Shanghai (CN); Zhiliu Zhang, Shanghai (CN)

(73) Assignee: GLAXSMITHKLINE LLC, Philadelphia, PA (US)

(21) Appl. No.: 13/375,595

(22) PCT Filed: Jun. 2, 2010
(86) PCT No.: PCT/US10/37011

§ 371 Date: Dec. 1, 2011

Related U.S. Application Data
(60) Provisional application No. 61/183,670, filed on Jun. 3, 2009.
(60) Provisional application No. 61/286,889, filed on Dec. 16, 2009.

(51) Int. Cl. A61K 31/444; C07D 487/04; C07D 401/14; C07D 487/08; C07D 498/04; A61K 31/437; A61K 31/4985; A61K 31/498; A61P 3/04; A61P 3/10; A61P 9/12; A61P 25/24; A61P 25/22; A61P 25/30; C07D 471/04

(52) U.S. Cl. 514/210.16; 546/113; 546/256; 544/349; 514/300; 514/333; 514/249

(57) ABSTRACT

The invention provides novel bis-pyridylpyridones which are antagonists at the melanin-concentrating hormone receptor 1 (MCHR1), pharmaceutical compositions containing them, processes for their preparation, and their use in therapy and for the treatment of obesity and diabetes.

### FIELD OF INVENTION

This invention relates to novel bis-pyridylpyridones which are antagonists at the melanin-concentrating hormone receptor 1 (MCHR1), to pharmaceutical compositions containing them, to processes for their preparation, and to their use in therapy for the treatment of obesity and diabetes.

### BACKGROUND OF THE INVENTION

Obesity is a medical condition that is reaching epidemic proportions among humans in a number of countries throughout the world. It is a condition that is also associated with or induces other diseases or conditions that disrupt life activities and lifestyles. Obesity is recognized as a serious risk factor for other diseases and conditions such as diabetes, hypertension, and arteriosclerosis. It is also known that increased body weight due to obesity can place a burden on joints, such as knee joints, causing arthritis, pain, and stiffness.

Because overeating and obesity have become such a problem in the general population, many individuals are now interested in losing weight, reducing weight, and maintaining a healthy body weight and desirable lifestyle.

It is known that melanin-concentrating hormone originates in the hypothalamus and has orexigenic action (see Nature, Vol. 396, p. 670 (1998), for example. There is an on-going need for the development of a melanin-concentrating hormone antagonist useful in the treatment of obesity and other associated or related diseases and conditions.

Accordingly, we have now found a novel group of bis-pyridylpyridones that exhibit a useful profile of activity as antagonists of the melanin-concentrating hormone receptor 1 (MCHR1).

### SUMMARY OF THE INVENTION

The present invention provides a compound of Formula (I),

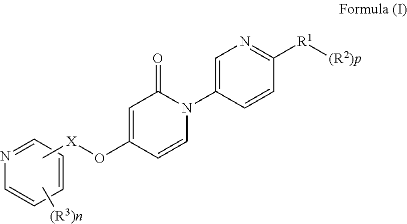

or a pharmaceutically acceptable salt thereof wherein:

R¹ is a saturated 6-12 membered heterocyclic ring system containing 1 or 2 ring nitrogen atoms and an optional ring oxygen atom, which ring system incorporates fused or bridged rings and which is attached to the pyridine ring via a nitrogen atom;

R² is selected from the group consisting of: hydrogen, C₁₋₆alkyl, hydroxyl, —C(O)NR^{a}R^{b}, —C(O)R^{a}, —SO₂R^{a}, —C(O)OR^{a}, oxo, —C(O)NR^{a}R^{b}, —NR^{a}R^{b}, —NR^{a}C(O)R^{b}, —NR^{a}C(O)OR^{b}, and —NR^{a}SO₂R^{b};

R^{a} is selected from the group consisting of: hydrogen, C₁₋₆alkyl, and C₃₋₆cycloalkyl;

R^{b} is selected from the group consisting of: hydrogen, C₁₋₆alkyl, C₃₋₆cycloalkyl, aryl, and heteroaryl;

or R^{a} and R^{b} together with the nitrogen to which they are attached form a heterocycloalkyl, and said heterocycloalkyl is optionally substituted with one, two, or three R^{c} groups;

R³ is H, F, Cl, C₁₋₃alkyl, cyclopropyl, C₁₋₃alkoxy, amino, C₁₋₃alkylamino, oxo, or CN;

R^{c} is H, F, Cl, C₁₋₆alkyl, C₃₋₆cycloalkyl, C₁₋₃alkoxy, amino, C₁₋₃alkylamino, oxo, or CN;

X is (CH₂)ₘ;

m is 0-2;

n is 0-3;

p is 0-3;

with the proviso that R² is not —NR^{a}COOH or —NR^{a}SO₂H.

There is also provided a pharmaceutical composition comprising a compound of Formula I or pharmaceutically acceptable salt thereof.

Further, there is provided a pharmaceutical composition comprising a compound of Formula I or salt thereof and one or more excipients.

There is still further provided a method of treatment comprising the administering to a mammal, particularly a human, a pharmaceutical composition comprising a compound of Formula I or pharmaceutically acceptable salt thereof and at least one excipient, wherein said treatment is for obesity, diabetes, depression, or anxiety.

Additionally, there is provided a compound of Formula I or pharmaceutically acceptable salt thereof for use as an active therapeutic substance (in therapy).

And, there is also provided a compound of Formula I or pharmaceutically acceptable salt thereof for use in the treatment of obesity, diabetes, depression, or anxiety in a mammal, especially a human.

A process for preparing a compound of Formula I or pharmaceutically acceptable salt thereof is also provided.

### DETAILED DESCRIPTION OF THE INVENTION

The present invention relates to compounds of Formula I as shown above.

The present invention also relates to a compound of Formula (I)(A)

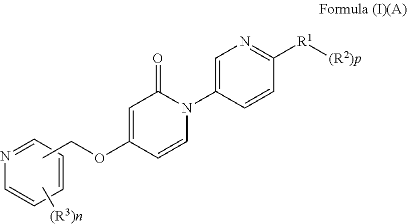

or a pharmaceutically acceptable salt thereof wherein:

R1 is a saturated 6-12 membered heterocyclic ring system containing 1 or 2 ring nitrogen atoms and an optional ring oxygen atom, which ring system incorporates fused or bridged rings and which is attached to the pyridine ring via a nitrogen atom;

R2 is selected from the group consisting of: hydrogen, C₁₋₆alkyl, hydroxyl, —C(O)NR^{a}R^{b}, —C(O)R^{a}, —SO₂R^{a}, —C(O)OR^{a}, oxo, —C(O)NR^{a}R^{b}, —NR^{a}R^{b}, —NR^{a}C(O)R^{b}, —NR^{a}C(O)OR^{b}, and —NR^{a}SO₂R^{b};

R^{a} is selected from the group consisting of: hydrogen, C₁₋₆alkyl, and C₃₋₆cycloalkyl;

R^{b} is selected from the group consisting of: hydrogen, C₁₋₆alkyl, and C₃₋₆cycloalkyl;

or R^{a} and R^{b} together with the nitrogen to which they are attached form a heterocycloalkyl, and said heterocycloalkyl is optionally substituted with one, two, or three R^{c} groups;

R³ is H, F, Cl, C₁₋₃alkyl, cyclopropyl, C₁₋₃alkoxy, amino, C₁₋₃alkylamino, oxo, or CN;

R^{c} is H, F, Cl, C₁₋₆alkyl, C₃₋₆cycloalkyl, C₁₋₃alkoxy, amino, C₁₋₃alkylamino, oxo, or CN;

n is 0-3;

p is 0-3;

with the proviso that R² is not —NR^{a}COOH or —NR^{a}SO₂H.

The present invention also relates to a compound of Formula (I)(B)

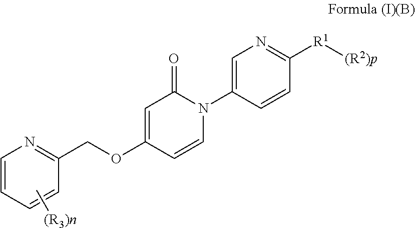

or a pharmaceutically acceptable salt thereof wherein:

R1 is a saturated 6-12 membered heterocyclic ring system containing 1 or 2 ring nitrogen atoms and an optional ring oxygen atom, which ring system incorporates fused or bridged rings and which is attached to the pyridine ring via a nitrogen atom;

R2 is selected from the group consisting of: hydrogen, C₁₋₆alkyl, hydroxyl, —C(O)NR^{a}R^{b}, —C(O)R^{a}, —SO₂R^{a}, —C(O)OR^{a}, oxo, —C(O)NR^{a}R^{b}, —NR^{a}R^{b}, —NR^{a}C(O)R^{b}, —NR^{a}C(O)OR^{b}, and —NR^{a}SO₂R^{b};

R^{a} is selected from the group consisting of: hydrogen, C₁₋₆alkyl, and C₃₋₆cycloalkyl;

R^{b} is selected from the group consisting of: hydrogen, C₁₋₆alkyl, and C₃₋₆cycloalkyl;

or R^{a} and R^{b} together with the nitrogen to which they are attached form a heterocycloalkyl, and said heterocycloalkyl is optionally substituted with one, two, or three R^{c} groups;

R³ is H, F, Cl, C₁₋₃alkyl, cyclopropyl, C₁₋₃alkoxy, amino, C₁₋₃alkylamino, oxo, or CN;

R^{c} is H, F, Cl, C₁₋₆alkyl, C₃₋₆cycloalkyl, C₁₋₃alkoxy, amino, C₁₋₃alkylamino, oxo, or CN;

n is 0-3;

p is 0-3;

with the proviso that R² is not —NR^{a}COOH or —NR^{a}SO₂H.

The present invention also relates to a compound of Formula (I)(C)

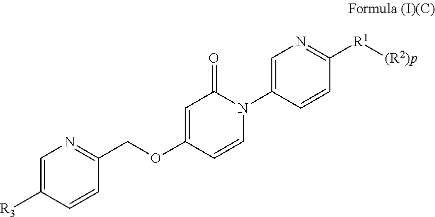

or a pharmaceutically acceptable salt thereof wherein:

R1 is a saturated 6-12 membered heterocyclic ring system containing 1 or 2 ring nitrogen atoms and an optional ring oxygen atom, which ring system incorporates fused or bridged rings and which is attached to the pyridine ring via a nitrogen atom;

R2 is selected from the group consisting of: hydrogen, C₁₋₆alkyl, hydroxyl, —C(O)NR^{a}R^{b}, —C(O)R^{a}, —SO₂R^{a}, —C(O)OR^{a}, oxo, —C(O)NR^{a}R^{b}, —NR^{a}R^{b}, —NR^{a}C(O)R^{b}, —NR^{a}C(O)OR^{b}, and —NR^{a}SO₂R^{b};

R^{a} is selected from the group consisting of: hydrogen, C₁₋₆alkyl, and C₃₋₆cycloalkyl;

R^{b} is selected from the group consisting of: hydrogen, C₁₋₆alkyl, and C₃₋₆cycloalkyl;

or R^{a} and R^{b} together with the nitrogen to which they are attached form a heterocycloalkyl, and said heterocycloalkyl is optionally substituted with one, two, or three R^{c} groups;

R³ is H, F, Cl, C₁₋₃alkyl, cyclopropyl, C₁₋₃alkoxy, amino, C₁₋₃alkylamino, oxo, or CN;

R^{c} is H, F, Cl, C₁₋₆alkyl, C₃₋₆cycloalkyl, C₁₋₃alkoxy, amino, C₁₋₃alkylamino, oxo, or CN;

p is 0-3;

with the proviso that R² is not —NR^{a}COOH or —NR^{a}SO₂H.

This invention also relates to compounds of Formula (I)(A), (I)(B) or (I)(C) wherein R1 is a bicyclic or bridged ring system selected from: octahydropyrrolopyrrole, octahydropyrrolopyridine, 3,6-diazabicyclo[3.2.0]hept-6-yl, 6-amino-3-azabicyclo[3.1.0]hex-3-yl, and azabicyclo[3.1.0]hexan-1-yl.

Preferred compounds of the invention are compounds of Formula (I)(A), (I)(B) or (I)(C) wherein R³ is F or Cl, R^{b} is selected from the group consisting of: hydrogen, C₁₋₆alkyl, and C₃₋₆cycloalkyl; m is 1; and n is 1.

Of the compounds of the present invention, the most preferred compounds are
4-{[(5-chloro-2-pyridinyl)methyl]oxy}-6′-(8-methyl-2,8-diazabicyclo[4.3.0]nonan-2-yl)-2H-1,3′-bipyridin-2-one,
4-{[(5-chloro-2-pyridinyl)methyl]oxy}-6′-(2,7-diazabicyclo[4.3.0]nonan-2-yl)-2H-1,3′-bipyridin-2-one,
4-{[(5-chloro-2-pyridinyl)methyl]oxy}-6′-(1-methylamino-5-methyl-azabicyclo[3.1.0]hexan-3-yl)-2H-1,3′-bipyridin-2-one,
4-{[(5-chloro-2-pyridinyl)methyl]oxy}-6′-(octahydropyrrolo[3,4-c]pyrrol-2-yl)-2H-1,3′-bipyridin-2-one,
4-{[(5-chloro-2-pyridinyl)methyl]oxy}-6′-(octahydropyrrolo[1,2-a]pyrazin-2-yl)-2H-1,3′-bipyridin-2-one,
4-{[(5-chloro-2-pyridinyl)methyl]oxy}-6′-(3,6-diazabicyclo[3.2.0]hept-6-yl)-2H-1,3′-bipyridin-2-one,
4-{[(5-chloro-2-pyridinyl)methyl]oxy}-6′-(3,6-diazabicyclo[3.2.0]hept-3-yl)-2H-1,3′-bipyridin-2-one,
4-{[(5-chloro-2-pyridinyl)methyl]oxy}-6′-(6-amino-3-azabicyclo[3.1.0]hex-3-yl)-2H-1,3′-bipyridin-2-one,
4-{[(5-chloro-2-pyridinyl)methyl]oxy}-6′-(octahydro-5H-pyrrolo[3,2-c]pyridin-5-yl)-2H-1,3′-bipyridin-2-one, and
4-{[(5-chloro-2-pyridinyl)methyl]oxy}-6′-(octahydro-6H-pyrrolo[3,4-b]pyridin-6-yl)-2H-1,3′-bipyridin-2-one.

It will be appreciated by those skilled in the art that the compound of the present invention may also be utilized in the form of a pharmaceutically acceptable salt thereof.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. In general, the salts are formed from pharmaceutically acceptable inorganic and organic acids. More specific examples of suitable acid salts include maleic, hydrochloric, hydrobromic, sulphuric, phosphoric, nitric, perchloric, fumic, acetic, propionic, succinic, glycolic, formic, lactic, aleic, tartaric, citric, palmoic, malonic, hydroxymaleic, phenylacetic, glutamic, benzoic, salicylic, fumaric, toluenesulfonic, methansulfonic (mesylate), naphthalene-2-sulfonic, benzenesulfonic, hydroxynaphthoic, hydroiodic, malic, teroic, tannic, and the like.

Other representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, oxalate, pamoate (embonate), palmitate, pantothenate, phosphate/diphosphate, polygalacturonate, salicylate, stearate, subacetate, succinate, sulfate, tannate, tartrate, teoclate, tosylate, triethiodide, and valerate salts.

Other salts, which are not pharmaceutically acceptable, may be useful in the preparation of compounds of this invention and these should be considered to form a further aspect of the invention. These salts, such as oxalic or trifluoroacetate, while not in themselves pharmaceutically acceptable, may be useful in the preparation of salts useful as intermediates in obtaining the compounds of the invention and their pharmaceutically acceptable salts.

The compound of Formula I or a salt thereof may exist in stereoisomeric forms (e.g., it contains one or more asymmetric carbon atoms). The individual stereoisomers (enantiomers and diastereomers) and mixtures of these are included within the scope of the present invention. The invention also covers the individual isomers of the compound or salt represented by Formula I as mixtures with isomers thereof in which one or more chiral centers are inverted. Likewise, it is understood that a compound or salt of Formula I may exist in tautomeric forms other than that shown in the formula and these are also included within the scope of the present invention. It is to be understood that the present invention includes all combinations and subsets of the particular groups defined hereinabove. The scope of the present invention includes mixtures of stereoisomers as well as purified enantiomers or enantiomerically/diastereomerically enriched mixtures. Also included within the scope of the invention are individual isomers of the compound represented by Formula I, as well as any wholly or partially equilibrated mixtures thereof. The present invention also includes the individual isomers of the compound or salt represented by the Formula I as well as mixtures with isomers thereof in which one or more chiral centers are inverted. It is to be understood that the present invention includes all combinations and subsets of the particular groups defined hereinabove.

### DEFINITIONS

Terms are used within their accepted meanings. The following definitions are meant to clarify, but not limit, the terms defined.

As used herein, the term “alkyl” (or “alkylene”) refers to a straight or branched chain alkyl, preferably having from one to twelve carbon atoms, which may be unsubstituted or substituted, saturated or unsaturated with multiple degrees of substitution included within the present invention. Suitable substituents are selected from the group consisting of halogen, amino, substituted amino, cyano, hydroxyl, alkoxy and alkylthio. Examples of “alkyl” as used herein include methyl, ethyl, propyl, isopropyl, isobutyl, n-butyl, t-butyl, isopentyl, n-pentyl, and the like, as well as substituted versions thereof.

As used herein, the term “cycloalkyl” refers to an unsubstituted or substituted mono- or polycyclic non-aromatic saturated ring, which optionally includes an alkylene linker through which the cycloalkyl may be attached. Exemplary “cycloalkyl” groups include, but are not limited to, cyclopropyl, cyclobutyl, cyclopentyl, cyclohexyl, cycloheptyl, and the like, as well as unsubstituted and substituted versions thereof.

As used herein, the term “alkoxy” refers to the group —OR^{a}, where R^{a} is C1-3alkyl or C3-7cycloalkyl as defined above.

As used herein, the term “heterocycle” or “heterocyclyl” refers to unsubstituted and substituted mono- or polycyclic non-aromatic ring system containing one or more heteroatoms. Preferred heteroatoms include N, O, and S, including N-oxides, sulfur oxides, and dioxides. Preferably the ring is three to eight-membered and is either fully saturated or has one or more degrees of unsaturation. Multiple degrees of substitution are included within the present definition. Examples of “heterocyclic” groups include, but are not limited to tetrahydrofuranyl, pyranyl, 1,4-dioxanyl, 1,3-dioxanyl, piperidinyl, pyrrolidinyl, morpholinyl, azetidinyl, piperazinyl, pyrrolidinonyl, piperazinonyl, pyrazolidinyl, and their various tautomers.

As used herein, the term “aryl”, unless otherwise defined, is meant aromatic, hydrocarbon, ring system. The ring system may be monocyclic or fused polycyclic (e.g., bicyclic, tricyclic, etc.), substituted or unsubstituted. In various embodiments, the monocyclic aryl ring is C5-C10, or C5-C7, or C5-C6, where these carbon numbers refer to the number of carbon atoms that form the ring system. A C6 ring system, i.e. a phenyl ring, is a suitable aryl group. In various embodiments, the polycyclic ring is a bicyclic aryl group, where suitable bicyclic aryl groups are C8-C12, or C9-C10. A naphthyl ring, which has 10 carbon atoms, is a suitable polycyclic aryl group. Suitable substituents for aryl are described below in the definition of “optionally substituted”.

As used herein, the term “heteroaryl”, unless otherwise defined, is meant an aromatic ring system containing carbon(s) and at least one heteroatom. Heteroaryl may be monocyclic or polycyclic, substituted or unsubstituted. A monocyclic heteroaryl group may have 1 to 4 heteroatoms in the ring, while a polycyclic heteroaryl may contain 1 to 10 hetero atoms. A polycyclic heteroaryl ring may contain fused, spiro or bridged ring junctions, for example, bicyclic heteroaryl is a polycyclic heteroaryl. Bicyclic heteroaryl rings may contain from 8 to 12 member atoms. Monocyclic heteroaryl rings may contain from 5 to 8 member atoms (carbons and heteroatoms). Exemplary heteroaryl groups include benzofuran, benzothiophene, furan, imidazole, indole, isothiazole, oxazole, pyrazine, pyrazole, pyridazine, pyridine, pyrimidine, pyrrole, quinoline, quinazoline, quinoxaline, thiazole, triazole and tetrazole and thiophene. Suitable substituents for heteroaryl are described below in the definition of “optionally substituted”.

As used herein, the term “cyano” refers to the group —CN.

As used herein, the term “acetyl” refers to the group —C(O)R^{b}, where R^{b} is C′-6alkyl, C3-7cycloalkyl, or heterocyclyl, as each is defined herein.

As used herein, the term “optionally” means that the subsequently described event(s) may or may not occur, and includes both event(s) that occur and event(s) that do not occur.

As used herein, unless otherwise defined, the phrase “optionally substituted” or variations thereof denote an optional substitution, including multiple degrees of substitution, with one or more substitutent group. The phrase should not be interpreted as duplicative of the substitutions herein described and depicted. Exemplary optional substituent groups include acyl, C1-6alkyl, alkylsulfonyl, alkoxy, alkoxycarbonyl, cyano, halogen, haloalkyl, hydroxyl, oxo, and nitro.

Fusion of the rings can occur in three ways:
1. Across a bond between two atoms—for example, decalin (also known as bicyclo[4.4.0]decane), has a C—C bond shared between two cyclohexane rings;
2. Across a sequence of atoms (bridgehead)—for example, norbornane (also known as bicyclo[2.2.1]heptane), can be viewed as a pair of cyclopentane rings that share three of the five carbon atoms;
3. At a single atom (spirocyclic, forming a spiro compound)
   Compounds of the present invention contain heterocycles that are fused in the first two ways above.

The compounds of this invention may be made by a variety of methods. Illustrative general synthetic methods are set out below and then specific compounds of the invention are prepared in the working examples.

In all of the schemes described below, protecting groups for sensitive or reactive groups are employed where necessary in accordance with general principles of synthetic chemistry. Protecting groups are manipulated according to standard methods of organic synthesis (T. W. Green and P. G. M. Wuts, (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention. Compounds of the invention can be readily prepared according to Schemes 1 and 2 by those skilled in the art.

Pyridone intermediates of the invention can be prepared as illustrated in Scheme 1. Briefly, alkoxyformylation of substituted bromopyridines (A) followed by reduction provided hydroxymethylpyridine intermediates (B). Reaction of intermediates (B) with 4-nitropyridine-1-oxide in the presence of sodium metal provided substituted hydroxymethyl ether intermediates (C). Treatment of intermediates (C) with trifluoroacetic anhydride (TFAA) or acetic anhydride (Ac₂O) provided the desired pyridone intermediates (D).

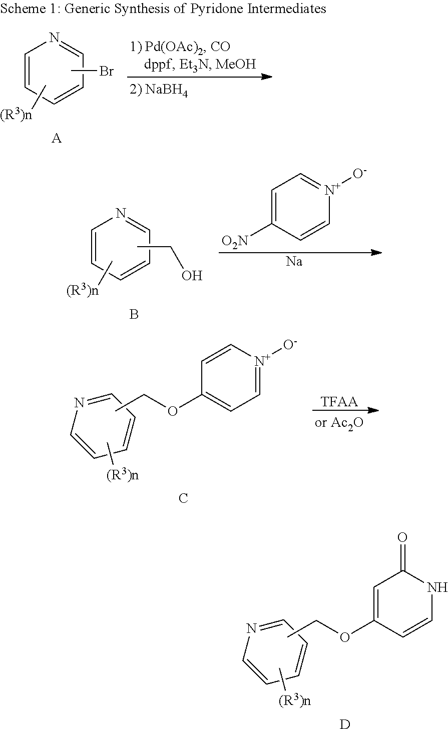

From these pyridone intermediates, compounds of the invention can be prepared as illustrated in Scheme 2. Briefly, reaction of substituted pyridone intermediates (D) with 2-amino-5-halo pyridines (E) provided 2-aminopyridine intermediates (F). Subsequent treatment of intermediates (F) with HF/pyridine, followed by treatment with NaNO₂, provided 2-fluoropyridine intermediates (G). Reaction of intermediates (G) with the amines encompassed within the scope of this invention (H, or appropriately functional-group-protected version thereof, with subsequent routine deprotection) provided Examples (I).

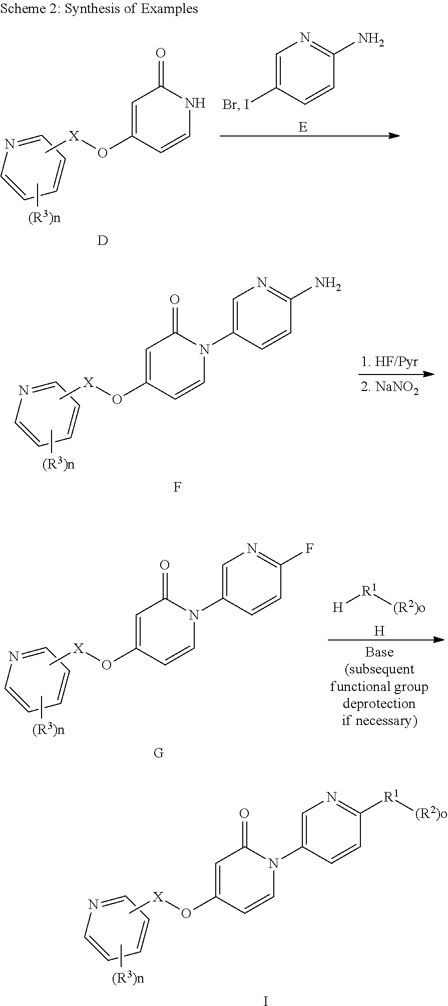

The requisite amines (and appropriately functional-group-protected versions thereof) utilized herein were purchased if available commercially, were synthesized as described in the literature or by routine modifications thereof known by those skilled in the art, or were synthesized by alternative procedures known by those skilled in the art.

The invention further provides a pharmaceutical composition (also referred to as pharmaceutical formulation) comprising a compound of Formula I or pharmaceutically acceptable salt, thereof and one or more excipients (also referred to as carriers and/or diluents in the pharmaceutical arts). The excipients are acceptable in the sense of being compatible with the other ingredients of the formulation and not deleterious to the recipient thereof (i.e., the patient).

In accordance with another aspect of the invention there is provided a process for the preparation of a pharmaceutical composition comprising mixing (or admixing) a compound of Formula I or salt thereof with at least one excipient.

Pharmaceutical compositions may be in unit dose form containing a predetermined amount of active ingredient per unit dose. Such a unit may contain a therapeutically effective dose of the compound of Formula I or salt thereof or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical compositions may be prepared by any of the methods well-known in the pharmacy art.

Pharmaceutical compositions may be adapted for administration by any appropriate route, for example, by oral (including buccal or sublingual), rectal, nasal, topical (including buccal, sublingual, or transdermal), vaginal, or parenteral (including subcutaneous, intramuscular, intravenous, or intradermal) routes. Such compositions may be prepared by any method known in the art of pharmacy, for example, by bringing into association the active ingredient with the excipient(s).

When adapted for oral administration, pharmaceutical compositions may be in discrete units such as tablets or capsules; powders or granules; solutions or suspensions in aqueous or non-aqueous liquids; edible foams or whips; oil-in-water liquid emulsions or water-in-oil liquid emulsions. The compound or salt thereof of the invention or the pharmaceutical composition of the invention may also be incorporated into a candy, a wafer, and/or tongue tape formulation for administration as a “quick-dissolve” medicine.

For instance, for oral administration in the form of a tablet or capsule, the active drug component can be combined with an oral, non-toxic pharmaceutically acceptable inert carrier such as ethanol, glycerol, water, and the like. Powders or granules are prepared by comminuting the compound to a suitable fine size and mixing with a similarly comminuted pharmaceutical carrier such as an edible carbohydrate, as, for example, starch or mannitol. Flavoring, preservative, dispersing, and coloring agents can also be present.

Capsules are made by preparing a powder mixture, as described above, and filling formed gelatin or non-gelatinous sheaths. Glidants and lubricants such as colloidal silica, talc, magnesium stearate, calcium stearate, solid polyethylene glycol can be added to the powder mixture before the filling operation. A disintegrating or solubilizing agent such as agar-agar, calcium carbonate, or sodium carbonate can also be added to improve the availability of the medicine when the capsule is ingested.

Moreover, when desired or necessary, suitable binders, lubricants, disintegrating agents, and coloring agents can also be incorporated into the mixture. Suitable binders include starch, gelatin, natural sugars, such as glucose or beta-lactose, corn sweeteners, natural and synthetic gums such as acacia, tragacanth, sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methylcellulose, agar, bentonite, xanthan gum, and the like.

Tablets are formulated, for example, by preparing a powder mixture, granulating or slugging, adding a lubricant and disintegrant, and pressing into tablets. A powder mixture is prepared by mixing the compound, suitably comminuted, with a diluent or base as described above, and optionally, with a binder such as carboxymethylcellulose, and aliginate, gelatin, or polyvinyl pyrrolidone, a solution retardant such as paraffin, a resorption accelerator such as a quaternary salt, and/or an absorption agent such as bentonite, kaolin, or dicalcium phosphate. The powder mixture can be granulated by wetting a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compound or salt of the present invention can also be combined with a free-flowing inert carrier and compressed into tablets directly without going through the granulating or slugging steps. A clear opaque protective coating consisting of a sealing coat of shellac, a coating of sugar, or polymeric material, and a polish coating of wax can be provided. Dyestuffs can be added to these coatings to distinguish different dosages.

Oral fluids such as solutions, syrups, and elixirs can be prepared in dosage unit form so that a given quantity contains a predetermined amount of active ingredient. Syrups can be prepared by dissolving the compound or salt thereof of the invention in a suitably flavoured aqueous solution, while elixirs are prepared through the use of a non-toxic alcoholic vehicle. Suspensions can be formulated by dispersing the compound or salt of the invention in a non-toxic vehicle. Solubilizers and emulsifiers, such as ethoxylated isostearyl alcohols and polyoxyethylene sorbitol ethers, preservatives, flavor additives such as peppermint oil, natural sweeteners, saccharin, or other artificial sweeteners, and the like, can also be added.

Where appropriate, dosage unit formulations for oral administration can be microencapsulated. The formulation can also be prepared to prolong or sustain the release as, for example, by coating or embedding particulate material in polymers, wax, or the like.

In the present invention, tablets and capsules are preferred for delivery of the pharmaceutical composition.

As used herein, the term “treatment” includes prophylaxis and refers to alleviating the specified condition, eliminating or reducing one or more symptoms of the condition, slowing or eliminating the progression of the condition, and preventing or delaying the reoccurrence of the condition in a previously afflicted or diagnosed patient or subject. Prophylaxis (or prevention or delay of disease onset) is typically accomplished by administering a drug in the same or similar manner as one would to a patient with the developed disease or condition.

The present invention provides a method of treatment in a mammal, especially a human, suffering from obesity, diabetes, hypertension, depression, anxiety, drug addiction, substance addiction, or a combination thereof. Such treatment comprises the step of administering a therapeutically effective amount of a compound of Formula I or salt thereof to said mammal, particularly a human. Treatment can also comprise the step of administering a therapeutically effective amount of a pharmaceutical composition containing a compound of Formula I or salt thereof to said mammal, particularly a human.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula I, as well as salts thereof, may be administered as the raw chemical. Additionally, the active ingredient may be presented as a pharmaceutical composition.

While it is possible that, for use in therapy, a therapeutically effective amount of a compound of Formula I or salt thereof may be administered as the raw chemical, it is typically presented as the active ingredient of a pharmaceutical composition or formulation.

The precise therapeutically effective amount of a compound or salt thereof of the invention will depend on a number of factors, including, but not limited to, the age and weight of the subject (patient) being treated, the precise disorder requiring treatment and its severity, the nature of the pharmaceutical formulation/composition, and route of administration, and will ultimately be at the discretion of the attending physician or veterinarian. Typically, a compound of Formula I or salt thereof will be given for the treatment in the range of about 0.1 to 100 mg/kg body weight of recipient (patient, mammal) per day and more usually in the range of 0.1 to 10 mg/kg body weight per day. Acceptable daily dosages may be from about 1 to about 1000 mg/day, and preferably from about 1 to about 100 mg/day. This amount may be given in a single dose per day or in a number (such as two, three, four, five, or more) of sub-doses per day such that the total daily dose is the same. An effective amount of a salt thereof may be determined as a proportion of the effective amount of the compound of Formula I per se. Similar dosages should be appropriate for treatment (including prophylaxis) of the other conditions referred herein for treatment. In general, determination of appropriate dosing can be readily arrived at by one skilled in medicine or the pharmacy art.

Additionally, the present invention comprises a compound of Formula I or salt thereof or a pharmaceutical composition thereof with at least one other anti-obesity drug and at least one anti-diabetes drug. Such anti-obesity drugs can include, for example, Metformin (or glucophage), CB1 receptor antagonists, GLP-1 agonists, opioid antagonists, and neurotransmitter reuptake inhibitors. When a compound of the invention is employed in combination with another anti-obesity drug or anti-diabetes drug, it is to be appreciated by those skilled in the art that the dose of each compound or drug of the combination may differ from that when the drug or compound is used alone. Appropriate doses will be readily appreciated and determined by those skilled in the art. The appropriate dose of the compound of Formula I or salt thereof and the other therapeutically active agent(s) and the relative timings of administration will be selected in order to achieve the desired combined therapeutic effect, and are with the expertise and discretion of the attending doctor or clinician.

### EXPERIMENTAL

The following examples are intended for illustration only and are not intended to limit the scope of the invention in any way, the invention being defined by the claims.

Unless otherwise noted, reagents are commercially available or are prepared according to procedures in the literature. The symbols and conventions used in the descriptions of processes, schemes, and examples are consistent with those used in the contemporary scientific literature, for example, the Journal of the American Chemical Society or the Journal of Biological Chemistry.

### I. Preparation of Intermediates

### Intermediate 1

### methyl 5-chloro-2-pyridinecarboxylate

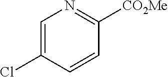

To a solution of 2-bromo-5-chloropyridine (30.0 g, 155.9 mmol) in MeOH (280 mL) was added Pd(OAc)₂ (3.5 g, 10.8 mmol), dppf (17.3 g, 37.96 mmol), Et₃N (42.0 mL, 312 mmol). The resulting mixture was stirred at 50° C. under a CO atmosphere (15 psi) for 24 h, then concentrated under reduced pressure to give crude residue. This residue was partitioned between EtOAc (3×500 mL) and water (300 mL). The combined organic layers were dried (Na₂SO₄) and evaporated. Flash chromatography of the residue over silica gel, by using 10:1 petroleum ether/EtOAc, afforded the title compound as a pale yellow solid (25 g, 93%): ¹H NMR (400 MHz, CDCl₃) δ ppm 8.60 (d, J=1.60 Hz, 1H), 8.01 (d, J=8.40 Hz, 1H), 7.75 (dd, J=8.40, 2.40 Hz, 1H), 3.92 (s, 3H).

### Intermediate 2

### (5-chloro-2-pyridinyl)methanol

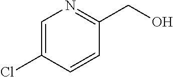

To a cooled (0° C.) solution of methyl 5-chloro-2-pyridinecarboxylate (43 g, 251 mmol) in methanol (400 mL) was added NaBH₄ (28.7 g, 754 mmol) in small portions over approximately 30 min. After addition, the reaction mixture was stirred at room temperature for 2 h, at which time TLC analysis showed the completion of the reaction. The reaction mixture was then concentrated under reduced pressure, and the residue was adjusted to pH 1 by adding 1N HCl. The resulting solution was extracted with EtOAc (3×300 mL). The combined organic layers were dried (Na₂SO₄) and evaporated. Flash chromatography of the residue over silica gel using 10:1 petroleum ether/EtOAc as eluent provided the title compound (36 g, 99%): ¹H NMR (400 MHz, CDCl₃) δ ppm 8.44 (d, J=1.60 Hz, 1H), 7.62 (dd, J=8.40, 2.40 Hz, 1H), 7.25 (d, J=8.40 Hz, 1H), 4.69 (s, 2H), 3.83 (s, 1H).

### Intermediate 3

### 4-{[(5-chloro-2-pyridinyl)methyl]oxy}pyridine-1-oxide

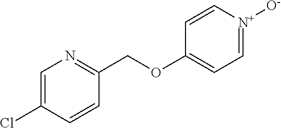

Sodium (7.5 g, 326 mmol) was added to a solution of (5-chloro-2-pyridinyl)methanol (36 g, 252 mmol) in THF (400 mL). After addition, the mixture was stirred at reflux for 16 h and then cooled to room temperature. To this mixture, a solution of 4-nitropyridine N-oxide (11.7 g, 84 mmol) in THF (100 mL) was added and the resulting mixture was stirred at room temperature for another 4 h. The mixture was filtered and the filtrate was concentrated under reduced pressure. Et₂O was added and a precipitate was formed. The precipitate was collected by filtration and washed with Et₂O (3×). This solid was dissolved in CH₂Cl₂ and filtered. The filtrate was dried (Na₂SO₄) and evaporated to give the title compound (9.7 g, 49%): ¹H NMR (400 MHz, CDCl₃) δ ppm 8.54 (d, J=0.80 Hz, 1H), 8.09 (m, 2H), 7.71 (dd, J=8.40, 2.40 Hz, 2H), 7.39 (dd, J=8.40, 0.40 Hz, 1H), 6.87 (m, 2H), 5.17 (s, 2H).

An alternative procedure was used for a larger-scale synthesis of the title compound. Thus, a stirred mixture of (5-chloro-2-pyridinyl)methanol (15.36 g, 107 mmol) and 4-nitropyridine 1-oxide (14.99 g, 107 mmol) in DCM (250 ml) cooled in an ice/water bath was charged with benzyltriethylammonium chloride (0.682 g, 3.00 mmol), and 9M NaOH (140 mL) was added dropwise via addition funnel. The mixture was stirred for 2.5 hours at room temperature with periodic checking by HPLC. The reaction mixture became a dark solution over this time period with easier stirring. LC/MS indicated that the reaction was complete. Water (300 mL) was added to the reaction and it quickly became an oily suspension. The reaction mixture was diluted with DCM and the organic layer was separated. The aqueous layer was extracted 3 more times with DCM, and the combined organic layers were washed with brine and dried over sodium sulfate. Concentration yielded a bright yellow solid, which was collected, washed with ether, and dried overnight (22.37 g, 88%): ES-LCMS m/z 237 (M+H)⁺.

### Intermediate 4

### 4-{[(5-chloro-2-pyridinyl)methyl]oxy}-2(1H)-pyridinone

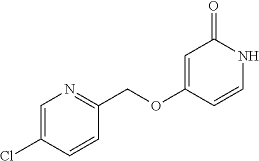

TFAA (9.7 g, 46.6 mmol) was added dropwise to a stirred and cooled (0° C.) solution of 4-{[(5-chloro-2-pyridinyl)methyl]oxy}pyridine-1-oxide (1.1 g, 4.7 mmol) and Et₃N (1.4 g, 14.0 mmol) in THF (15 mL). The reaction mixture was stirred at room temperature for 16 h, at which time TLC analysis showed almost completion of the reaction. The reaction mixture was diluted with water, and then extracted with CH₂Cl₂ (3×). The combined organic layers were washed with water, 1 N NaOH, brine, dried and concentrated in vacuo. The residue solid was triturated with ether to give the title compound (850 mg, 77%): ¹HNMR (400 MHz, DMSO-d₆) δ ppm 11.11 (s, 1H), 8.61 (s, 1H), 7.96 (d, J=6.00 Hz, 1H), 7.52 (d, J=8.40 Hz, 1H), 7.23 (d, J=7.60 Hz, 1H), 5.92 (d, J=4.80 Hz, 1H), 5.73 (s, 1H), 5.10 (s, 2H); ES-LCMS m/z 237 (M+H)⁺.

An alternative procedure was used for a larger-scale synthesis of the title compound. Thus, 4-{[(5-chloro-2-pyridinyl)methyl]oxy}pyridine-1-oxide (25 g, 106 mmol) and triethylamine (44.2 mL, 317 mmol) were allowed to stir in 300 mL of THF while cooling in an ice bath. Trifluoroacetic anhydride (224 mL, 1585 mmol) was added dropwise via addition funnel. The reaction mixture was allowed to stir an additional 15 min at ice bath temperature, and then warmed to room temperature. The reaction was allowed to stir overnight at room temperature. The next morning, LC/MS indicated that the reaction was complete. The reaction was poured over ice, and the resulting solution was extracted with DCM (4×100 mL). The organic layers were combined, washed with water, 1N NaOH, saturated brine solution, dried over sodium sulfate, and concentrated. The resulting solid was purified via chromatography using a gradient of (0-100% EtOAC/hexanes over a 30 minute run), to provide the title compound as a white solid (15 g, 60%).

A second alternative procedure was used on a similar scale. Thus, a solution of 4-{[(5-chloro-2-pyridinyl)methyl]oxy}pyridine-1-oxide (21 g, 90 mmol) in anhydrous Ac₂O (210 mL) was heated at reflux for 2 h. The mixture was concentrated under reduced pressure. The residue was dissolved in EtOAc/MeOH (5:1 v/v, 400 mL) and the mixture was refluxed for another 2 h. The solvent was removed, and the residue was dissolved in EtOAc (200 mL). The mixture was left standing overnight. The mixture was filtered, and the filter cake was washed with EtOAc to give the title compound as a black solid (15 g, 75%).

### Intermediate 5

### 4-{[(5-chloro-2-pyridinyl)methyl]oxy}-6′-(amino)-2H-1,3′-bipyridin-2-one

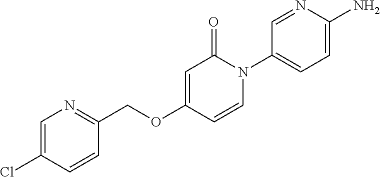

To a solution of 4-{[(5-chloro-2-pyridinyl)methyl]oxy}-2(1H)-pyridinone (9 g, 38 mmol) in anhydrous DMF (250 mL) was added 2-amino-5-iodo pyridine (9.18 g, 41.7 mmol), CuI (1.5 g, 7.56 mmol), K₂CO₃ (15.7 g, 114 mmol), and 8-hydroxyquinoline (0.9 g, 7.2 mmol), and the mixture was heated at 120° C. for 12 h. After LC-MS showed the stating material was consumed, the solvent was removed in vacuo to give the crude product, which was purified by column chromatography (3:1 EA/PE, to EA, to 10:1 DCM/MeOH, to MeOH) to afford the title compound (8.0 g, 71.9%): ¹H NMR (400 MHz, CD₃OD) δ ppm 8.51 (d, J=1.60 Hz, 1H), 7.83-7.87 (m, 2H), 7.52 (d, J=8.40 Hz, 1H), 7.45 (d, J=7.60 Hz, 1H), 7.39 (d, J=8.80 Hz, 1H), 6.61 (t, J=8.00 Hz, 1H), 6.24 (t, J=8.00 Hz, 1H), 6.00 (d, J=2.80 Hz, 1H), 5.17 (s, 2H); ES-LCMS m/z 329 (M+H)⁺.

### Intermediate 6

### 4-{[(5-chloro-2-pyridinyl)methyl]oxy}-6′-(fluoro)-2H-1,3′-bipyridin-2-one

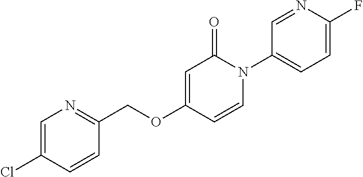

To a solution of HF/pyridine (50 mL) in pyridine (50 mL) in an ice bath was added 4-{[(5-chloro-2-pyridinyl)methyl]oxy}-6′-(amino)-2H-1,3′-bipyridin-2-one (5.0 g, 15.2 mmol). After stirring at room temperature for 30 min, the mixture was cooled at −20° C. NaNO₂ (1.5 g, 20 mmol) was added, and the reaction mixture was stirred at room temperature for 2 h. After TLC showed the starting material was completely consumed, the mixture was poured into saturated aqueous K₂CO₃ solution (200 mL) at 0° C. with stirring. The mixture was extracted with EA (3×800 mL), and the combined organic layer was dried over MgSO₄, and concentrated to give 4-{[(5-chloro-2-pyridinyl)methyl]oxy}-6′-(fluoro)-2H-1,3′-bipyridin-2-one (4.7 g, 93%): ¹H NMR (400 MHz, CD₃OD) δ ppm 8.57 (d, J=2.40 Hz, 1H), 8.24 (d, J=2.00 Hz, 1H), 8.03 (d, J=8.00 Hz, 1H), 7.91 (dd, J=7.60, 2.40 Hz, 1H), 7.58 (d, J=7.60 Hz, 2H), 7.20 (dd, J=7.20, 2.80 Hz, 1H), 6.32 (dd, J=7.60, 2.40 Hz, 1H), 6.08 (d, J=2.40 Hz, 1H), 5.23 (s, 2H); ES-LCMS m/z 332 (M+H)⁺.

### Intermediate 7

### tert-butyl 5-methyl-3-azabicyclo[3.1.0]hexan-1-yl N-methylcarbamate

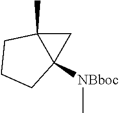

### Step 1: 2-[1(R)-phenylethylamino]acetonitrile

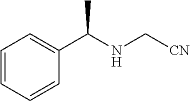

To a solution of (R)-1-phenylethylamine (70.00 g, 577.65 mmol) in MeCN (1.4 L) was added i-Pr₂Et (149.32 g, 1.16 mol) and BrCH₂CN (76.22 g, 635.42 mmol) and the mixture was stirred for 5 h at room temperature. The reaction mixture was concentrated, and this residue was combined with the residue from an identical reaction run in parallel, and the combined residues were portioned between saturated aqueous NaHCO₃ (2 L), brine (2 L), and CH₂Cl₂ (2×3 L). The combined organic extracts were washed with brine (2×3 L), dried (Na₂SO₄), filtered and concentrated to afford 2-[1(R)-phenylethylamino]acetonitrile as a yellow oil (181 g, 98%): ¹H NMR (400 MHz, CDCl₃) δ ppm 7.26-7.36 (m, 5H), 4.02-4.06 (m, 1H), 3.55-3.68 (m, 1H), 3.26 (d, J=17.2 Hz, 1H), 2.11 (m, 1H), 1.40-1.42 (m, 3H); ES-LCMS m/z 201 (M+H)⁺.

### Step 2: 2-{N—[(R)-1-phenylethyl]-N-[2-methylallyl]amino}acetonitrile

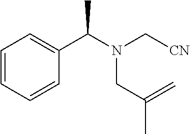

A mixture of 2-[1(R)-phenylethylamino]acetonitrile (30.00 g, 187.25 mmol), 3-iodo-2-methylprop-1-ene (51.12 g, 280.87 mmol) and K₂CO₃ (38.82 g, 280.87 mmol) was refluxed in anhydrous acetonitrile (400 mL) for 18 h. The mixture was filtered and the filtrate was concentrated to give the residue, which was purified by column chromatography on silica [PE (1 L) to 30:1 PE/EA (6 L)] to give 2-{N—[(R)-1-phenylethyl]-N-[2-methylallyl]amino}acetonitrile as a yellow oil (36 g, 90%): ¹H NMR (400 MHz, CDCl₃) δ ppm 7.16-7.29 (m, 5H), 4.93 (d, J=0.8 Hz, 2H), 3.60-3.63 (m, 1H), 3.49 (d, J=16.4 Hz, 1H), 3.34 (d, J=17.2 Hz, 1H), 2.93-3.03 (m, 2H), 1.61 (s, 3H), 1.17 (d, J=7.2 Hz, 3H); ES-LCMS m/z 215 (M+H)⁺.

### Step 3: 5-methyl-3-[1(R)-phenylethyl]-3-azabicyclo[3.1.0]hexan-1-amine

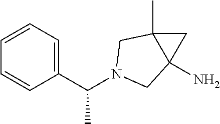

To a solution of 2-{N—[(R)-1-phenylethyl]-N-[2-methylallyl]amino}acetonitrile (10.00 g, 46.66 mmol) in dry Et₂O (300 mL) was added Ti(OiPr)₄ (14.59 g, 51.33 mmol) and cyclopentylmagnesium bromide (17.79 g, 102.66 mmol) successively at room temperature and the mixture was stirred for 0.5 h. BF₃.Et₂O (13.25 g, 93.32 mmol) was added and the mixture was stirred for further 0.5 h. The mixture was adjusted to pH 8 with aqueous 10% NaOH solution and filtered. The filtrate was condensed to give 5-methyl-3-[1(R)-phenylethyl]-3-azabicyclo[3.1.0]hexan-1-amine, which was used in the next step without further purification: ES-LCMS m/z 217 (M+H)⁺.

### Step 4: tert-butyl 5-methyl-3-[1(R)-phenylethyl]-3-azabicyclo[3.1.0]hexan-1-yl carbamate

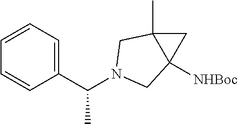

To a solution of 5-methyl-3-[1(R)-phenylethyl]-3-azabicyclo[3.1.0]hexan-1-amine (10.00 g, 46.23 mmol) in H₂O (300 mL) was added aqueous 10% NaOH solution (100 mL) and Boc₂O (11.10 g, 50.85 mmol) and the mixture was stirred at room temperature for 21 h. After the reaction was over, the mixture was extracted with ethyl acetate (3×400 mL). The combined organic layer was dried (Na₂SO₄) and concentrated to give the residue, which was purified by column chromatography on silica [PE (2 L) to 50:1 PE/EA (8 L) to 30:1 PE/EA (5 L)] to give tert-butyl 5-methyl-3-[1(R)-phenylethyl]-3-azabicyclo[3.1.0]hexan-1-yl carbamate as a yellow oil (2.3 g, 15%): ¹H NMR (400 MHz, CDCl₃) δ ppm 7.13-7.27 (m, 5H), 4.45-498 (m, 1H), 2.85-3.39 (m, 2H), 2.21-2.65 (m, 2H), 1.58-1.75 (m, 2H), 0.92-1.49 (m, 15H); ES-LCMS m/z 317 (M+H)⁺.

### Step 5: tert-butyl 5-methyl-3-[1(R)-phenylethyl]-3-azabicyclo[3.1.0]hexan-1-yl N-methylcarbamate

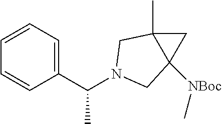

To a solution of tert-butyl 5-methyl-3-[1(R)-phenylethyl]-3-azabicyclo[3.1.0]hexan-1-yl carbamate (1.00 g, 3.16 mmol) in anhydrous THF (20 mL) at 0° C. was added NaH (150 mg, 6.32 mmol) portionwise. After the mixture was stirred for 30 min, a solution of MeI (493 mg, 3.48 mmol) in THF (2 mL) was added dropwise. The mixture was stirred at room temperature. for 18 hours. The reaction was quenched with H₂O (15 mL) and the mixture was extracted with CH₂Cl₂ (3×15 mL). The organic layers were combined, dried (Na₂SO₄) and concentrated. The residue was purified by preparative HPLC to give tert-butyl 5-methyl-3-[1(R)-phenylethyl]-3-azabicyclo[3.1.0]hexan-1-yl N-methylcarbamate as a white solid (2.05 g, 56%): ¹H NMR (400 MHz, CDCl₃) δ ppm 7.15-7.26 (m, 5H), 3.25-3.26 (m, 1H), 2.82 (s, 2H), 2.71 (s, 2H), 2.49-2.52 (m, 2H), 1.26-1.41 (m, 15H); ES-LCMS m/z 331 (M+H)⁺.

### Step 6: tert-butyl 5-methyl-3-azabicyclo[3.1.0]hexan-1-yl N-methylcarbamate

To a solution of tert-butyl 5-methyl-3-[1(R)-phenylethyl]-3-azabicyclo[3.1.0]hexan-1-yl N-methylcarbamate (500 mg, 1.51 mmol) in MeOH (50 mL) was added Pd(OH)₂ (50 mg, 0.36 mmol). The mixture was degassed and stirred under H₂ (30 psi) atmosphere for 25 h at room temperature. The mixture was filtered, and the filtrate was concentrated in vacuo to provide tert-butyl 5-methyl-3-azabicyclo[3.1.0]hexan-1-yl N-methylcarbamate (340 mg, 99%) as a white solid: ¹H NMR (400 MHz, CD₃OD) δ ppm 8.54 (s, 3H), 3.30-3.34 (m, 4H), 2.82-2.88 (m, 3H), 1.47 (m, 9H), 1.17-1.19 (m, 3H); ES-LCMS m/z 227 (M+H)⁺.

### II. Preparation of Compounds of the Invention

### Example 1

### 4-{[(5-chloro-2-pyridinyl)methyl]oxy}-6′-{(4R,9R)-8-methyl-2,8-diazabicyclo[4.3.0]nonan-2-yl)}-2H-1,3′-bipyridin-2-one

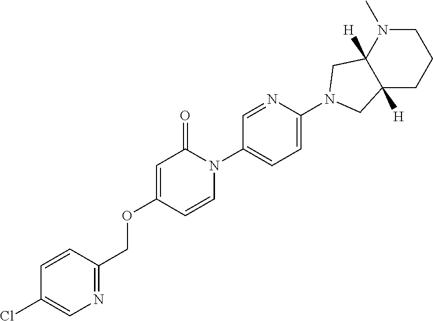

(4R,9R)-8-Methyl-2,8-diazabicyclo[4.3.0]nonane (42 mg, 0.3 mmol), potassium carbonate (104 mg, 0.75 mmol) and 4-{[(5-chloro-2-pyridinyl)methyl]oxy}-6′-(fluoro)-2H-1,3′-bipyridin-2-one (100 mg, 0.3 mmol) were dissolved in DMF (2 mL) and the mixture was stirred at 110° C. for 18 h. The solvent was removed in vacuo to give the crude product, which was purified by preparative HPLC to afford the target compound (34.82 mg, 25.7%): ¹H NMR (400 MHz, MeOH-d₄) δ 8.59 (d, J=2.40 Hz, 1H), 8.14 (d, J=2.40 Hz, 1H), 7.93 (dd, J=8.4, 2.4 Hz, 1H), 7.82-7.79 (m, 1H), 7.59 (d, J=8.4 Hz, 1H), 7.53 (d, J=6.4 Hz, 1H), 6.90 (m, 1H), 6.34-6.32 (m, 1H), 6.09 (d, J=2.8 Hz, 1H), 5.25 (s, 2H), 4.25 (m, 1H), 3.90 (m, 2H), 3.76 (m, 1H), 3.60-3.48 (m, 2H), 3.28-2.97 (m, 5H), 1.98 (m, 4H); ES-LCMS m/z 452 (M+H)⁺.

### Example 2

### 4-{[(5-chloro-2-pyridinyl)methyl]oxy}-6′-(2,7-diazabicyclo[4.3.0]nonan-2-yl)-2H-1,3′-bipyridin-2-one

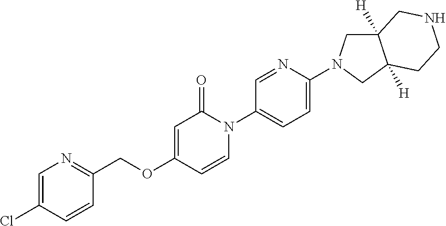

4-{[(5-chloro-2-pyridinyl)methyl]oxy}-6′-(fluoro)-2H-1,3′-bipyridin-2-one (50 mg, 0.15 mmol), tert-butyl 2,7-diazabicyclo[4.3.0]nonan-7-yl carboxylate (37.47 mg, 0.16 mmol) and K₂CO₃ (41.4 mg, 0.30 mmol) were dissolved in DMF (2 mL). Then the mixture was stirred at 110° C. for 18 h. After LC-MS showed that the stating material was consumed, the solvent was removed in vacuo to give crude 4-{[(5-chloro-2-pyridinyl)methyl]oxy}-6′-(7-tert-butyloxycarbonyl-2,7-diazabicyclo[4.3.0]nonan-2-yl)-2H-1,3′-bipyridin-2-one (20 mg, 35%): ¹H NMR (400 MHz, MeOH-d₄) δ 8.60 (d, J=2.40 Hz, 2H), 8.06 (d, J=2.80 Hz, 1H), 7.42 (m, 2H), 7.22 (m, 2H), 7.08 (m, 1H), 6.41 (d, J=8.80 Hz, 1H), 5.17 (s, 2H), 3.72 (m, 2H), 3.53 (m, 2H), 3.44 (m, 2H), 3.18 (m, 2H), 2.48 (m, 2H), 1.83 (m, 2 H), 1.41 (s, 9H); ES-LCMS m/z 538 (M+H)⁺.

4-{[(5-Chloro-2-pyridinyl)methyl]oxy}-6′-(7-tert-butyloxycarbonyl-2,7-diazabicyclo[4.3.0]nonan-2-yl)-2H-1,3′-bipyridin-2-one (20 mg, 0.037 mmol) was dissolved in 4N HCl/MeOH (20 mL). Then the mixture was stirred at 0° C. for 0.5 h. After TLC showed that the stating material was consumed, the solvent was removed in vacuo to give the crude product, which was purified by preparative HPLC to afford the title compound (16.23 mg, 100%): ¹H NMR (400 MHz, MeOH-d₄) δ ppm 8.56 (s, 1H), 8.01 (d, J=2.4 Hz, 1H), 7.91 (dd, J=8.4, 2.4 Hz, 1H), 7.85 (dd, J=9.4, 2.6 Hz, 1H), 7.53 (m, 2H), 6.95 (d, J=9.6 Hz, 1H), 6.32 (dd, J=7.6, 2.8 Hz, 1H), 6.07 (d, J=2.8 Hz, 1H), 5.23 (s, 2H), 3.72-3.81 (m, 2H), 3.58-3.64 (m, 2H), 3.32-3.41 (m, 1H), 3.13-3.31 (m, 3H), 2.72-2.89 (m, 2H), 2.01-2.12 (m, 1H), 1.80-1.92 (m, 1H); ES-LCMS m/z 438 (M+H)⁺.

### Example 3

### 4-{[(5-chloro-2-pyridinyl)methyl]oxy}-6′-(octahydro-5H-pyrrolo[3,4-b]pyrrol-5-yl)-2H-1,3′-bipyridin-2-one

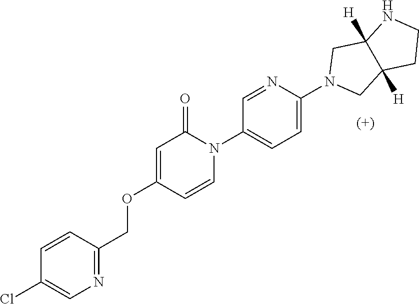

1-Benzyl-octahydropyrrolo[3,4-b]pyrrole (40 mg, 0.202 mmol), potassium carbonate (104 mg, 0.75 mmol) and 4-{[(5-chloro-2-pyridinyl)methyl]oxy}-6′-(fluoro)-2H-1,3′-bipyridin-2-one (50 mg, 0.176 mmol) were dissolved in DMF (2 mL) and the mixture was stirred at 110° C. for 18 h. After LC-MS showed that the starting material was consumed, the solvent was removed in vacuo to give 4-{[(5-chloro-2-pyridinyl)methyl]oxy}-6′-(1-benzyl-octahydropyrrolo[3,4-b]pyrrol-5-yl)-2H-1,3′-bipyridin-2-one, which was used in the next step without further purification (90 mg, 85.7%): ¹H NMR (400 MHz, CDCl₃) δ 8.51 (d, J=2.0 Hz, 1H), 7.99 (d, J=2.4 Hz, 1H), 7.65 (dd, J=8.4, 2.4 Hz, 1H), 7.40 (dd, J=9.2, 2.8 Hz, 1H), 7.35 (d, J=8.4 Hz, 1H), 7.24 (m, 5H), 7.16 (m, 1H); 6.32 (d, J=8.8 Hz, 1H), 6.02 (d, J=7.6 Hz, 1H), 6.02 (d, J=2.4 Hz, 1H), 5.94 (d, J=2.8 Hz, 1H); 5.08 (s, 2H), 3.86 (m, 1H), 3.59 (m, 2H), 3.34 (m, 3H), 3.19 (m, 1H), 2.92-2.85 (m, 2H), 2.29 (m, 1H), 2.02 (m, 1H); 1.04 (m, 1H); ES-LCMS m/z 514 (M+H)⁺.

To a solution of 4-{[(5-chloro-2-pyridinyl)methyl]oxy}-6′-(1-benzyl-octahydropyrrolo[3,4-b]pyrrol-5-yl)-2H-1,3′-bipyridin-2-one (90 mg, 0.175 mmol) in anhydrous 1,2-dichloro-ethane (20 mL) at 0° C. was added 1-chloroethylchloroformate (124 mg, 0.875 mmol) dropwise, and the mixture was stirred under reflux for 4 h. After TLC showed that the starting material was consumed, the mixture was evaporated. The residue was dried in vacuo and re-dissolved in anhydrous MeOH (20 mL). The mixture was stirred under reflux for 3 h. After LC-MS showed that the starting material was consumed, the solvent was removed in vacuo to give the crude product, which was purified by preparative HPLC to afford the title compound (9.49 mg, 15.7%): ¹H NMR (400 MHz, MeOH-d₄) δ 8.58 (d, J=2.4 Hz, 1H), 8.17 (d, J=2.4 Hz, 1H), 7.92 (dd, J=8.4, 2.4 Hz, 1H), 7.88-7.85 (m, 1H), 7.60-7.53 (m, 2H), 6.99 (d, J=9.2 Hz, 1H), 6.33 (dd, J=7.6, 2.4 Hz, 1H), 6.08 (d, J=2.8 Hz, 1H), 5.24 (s, 2H), 4.50 (m, 1H), 4.15-4.11 (m, 1H), 3.92-3.81 (m, 2H), 3.62-3.61 (m, 1H), 3.46-3.43 (m, 3H), 2.38 (m, 1H), 2.11 (m, 1H); ES-LCMS m/z 423 (M+H)⁺.

Examples 4-32 of the Compounds of Formula I were prepared by the methods described above for Examples 1-3, or routine variations thereof, starting from the requisite 6′-halopyridine and amine (or appropriately functional-group-protected version thereof, with subsequent routine deprotection). The requisite amines (and appropriately functional-group-protected versions thereof) utilized herein were purchased if available commercially, were synthesized as described in the literature or by routine modifications thereof known by those skilled in the art, or were synthesized by alternative procedures known by those skilled in the art.

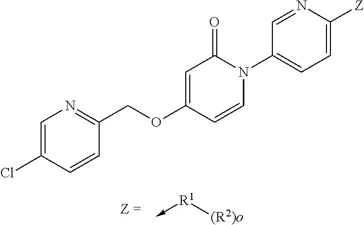

| Example | | | |
|---|---|---|---|
| (preparation) | Z | NMR Data | (M + H)⁺ |
| 4 (as Example 2) | 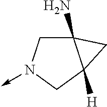 | ¹H NMR (400 MHz, MeOH- d₄) δ 8.52 (d, J = 2.4 Hz, 1H), 8.00 (d, J = 2.4 Hz, 1H), 7.86 (dd, J = 8.4, 2.4 Hz, 1H), 7.55 (m, 2H), 7.45 (d, J = 7.6 Hz, 1H), 6.61 (d, J = 9.4 Hz, 1H), 6.25 (dd, J = 7.6, 2.4 Hz, 1H), 6.02 (d, J = 2.8 Hz, 1H), 5.18 (s, 2H), 4.10 (d, J = 10 Hz, 1H), 3.68 (d, J = 10 Hz, 1H), 3.57 (m, 2H), 2.09 (m, 1H), 1.33 (m, 1H), 0.92 (m, 1H) | ES-LCMS m/z 410 |
| 5 (as Example 2) | 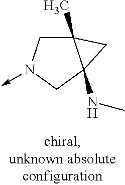 | ¹H NMR (400 MHz, MeOH- d₄) δ 8.57 (s, 1H), 8.13 (d, J = 2.4 Hz, 1H), 7.81-7.93 (m, 2H), 7.53-7.59 (m, 2H), 6.93 (d, J = 8.0 Hz, 1H), 6.32 (dd, J = 7.6, 2.4 Hz, 1H), 6.07 (d, J = 2.4 Hz, 1H), 5.24 (s, 2H), 4.23 (d, J = 10.4 Hz, 1H), 3.86 (m, 2H), 3.57 (d, J = 10.1 Hz, 1H), 3.29 (m, 2H), 2.93 (s, 3H), 1.53 (s, 3H) | ES-LCMS m/z 438 |
| 6 (as Example 2) | 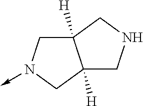 | ¹H NMR (400 MHz, MeOH- d₄) δ 8.51 (d, J = 2.4 Hz, 1H), 8.09 (d, J = 2.4 Hz, 1H), 7.95 (d, J = 2.40 Hz, 1H), 7.84 (d, J = 2.40 Hz, 1H), 7.50 (m, 2H), 7.02 (d, J = 9.60 Hz, 1H), 6.28 (dd, J = 7.6, 2.4 Hz 1H), 6.03 (d, J = 2.8 Hz, 1H), 5.18 (s, 2H), 3.83 (m, 2H), 3.55-3.65 (m, 5H), 3.23-3.33 (m, 3H) | ES-LCMS m/z 424 |
| 7 (as Example 2) | 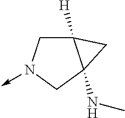 | ¹H NMR (400 MHz, MeOH- d₄) δ 8.48 (d, J = 2.4 Hz, 1H), 7.90 (d, J = 2.4 Hz, 1H), 7.82 (m, 1H), 7.51 (s, 1H), 7.48-7.50 (m, 2H), 6.49 (d, J = 8.8 Hz, 1H), 6.19-6.22 (m, 1H), 5.97 (d, J = 2.4 Hz, 1H), 5.14 (s, 2H), 3.87 (d, J = 9.2 Hz, 1H), 3.44-3.56 (m, 3H), 3.20-3.25 (m, 2H), 2.46 (s, 3H), 1.73-1.77 (m, 1H) | ES-LCMS m/z 424 |
| 8 (as Example 2) | 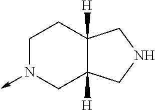 | ¹H NMR (400 MHz, MeOH- d₄) δ 8.58 (d, J = 2.0 Hz, 1H), 8.08 (d, J = 2.4 Hz, 1H), 7.91 (dd, J = 8.4, 2.4 Hz, 1H), 7.69 (dd, J = 9.2, 2.8 Hz, 1H), 7.58 (d, J = 8.4 Hz, 1H), 7.42 (d, J = 7.6 Hz, 1H), 7.05 (d, J = 9.2 Hz, 1H), 6.32 (dd, J = 7.6, 2.8 Hz, 2H), 6.08 (d, J = 2.8 Hz, 1H), 5.24 (s, 1H), 3.71-3.86 (m, 2H), 3.72 (dd, J = 14, 4.4 Hz, 1H), 3.48-3.62 (m, 2H), 3.41-3.46 (m, 1H), 3.20 (dd, J = 11.8, 4.6 Hz, 1H), 3.02 (m, 1H), 2.65-2.69 (m, 2H), 1.94-2.05 (m, 1H), 1.62-1.71 (m, 1H) | ES-LCMS m/z 438 |
| 9 (as Example 2) | 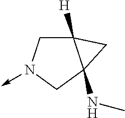 | ¹H NMR (400 MHz, MeOH- d₄) δ 8.58 (d, J = 2.4 Hz, 1H), 8.14 (d, J = 2.4 Hz, 1H), 7.93 (d, J = 2.4 Hz, 1H), 7.81-7.84 (m, 1H), 7.54-7.60 (m, 2H), 6.91-6.93 (d, J = 9.2 Hz, 1H), 6.32-6.35 (dd, J = 7.6, 2.4, 1H), 6.09 (d, J = 2.8 Hz, 1H), 5.24 (s, 2H), 4.23 (d J = 10.4 Hz, 1H), 3.86- 3.89 (m, 1H), 3.80 (s, 2H), 3.30-3.31 (m, 2H), 2.88 (s, 3H), 2.22-2.33 (m, 1H) | ES-LCMS m/z 424 |
| 10 (as Example 2) | 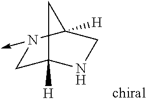 | ¹H NMR (400 MHz, MeOH- d₄) δ 8.56 (d, J = 1.2 Hz, 1H), 8.08 (d, J = 2.0 Hz, 1H), 7.91 (dd, J = 8.4, 2.4 Hz, 1H), 7.65 (dd, J = 9.0, 2.6 Hz, 1H), 7.58 (d, J = 8.4 Hz, 1H), 7.51 (d, J = 7.6 Hz, 1H), 6.76 (m, 1H), 6.31 (dd, J = 7.6, 2.4 Hz, 1H), 6.06 (d, J = 2.4 Hz, 1H), 5.23 (s, 2H), 5.02 (s, 1H), 4.88 (s, 1H), 3.75 (dd, J = 11.2, 2.0 Hz, 1H), 3.63 (m, 1H), 3.40 (m, 2H), 2.30 (m, 1H), 2.10 (m, 1H) | ES-LCMS m/z 410 |
| 11 (as Example 2) | 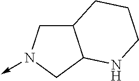 | ¹H NMR (400 MHz, MeOH- d₄) δ 8.59 (d, J = 2.00 Hz, 1H), 8.12 (d, J = 2.40 Hz, 1H), 7.93 (d, J = 2.40 Hz, 1H), 7.72 (s, 1H), 7.59 (d, J = 7.60 Hz, 1H), 7.54 (dd, J = 7.20, 2.80 Hz, 1H), 6.83 (d, J = 9.2 Hz, 1H), 6.34 (t, J = 5.0 Hz, 1H), 6.10 (d, J = 2.40, 1H), 5.26 (s, 2H), 4.01 (d, J = 4.40, 1H), 3.89 (d, J = 1.6, 1H), 3.78 (t, J = 10.40, 1H), 3.56 (t, J = 10.40, 1H), 3.38- 3.31 (m, 1H), 3.12-3.05 (m, 1H), 2.96-2.92 (m, 1H), 2.01- 1.86 (m, 4H) | ES-LCMS m/z 438 |
| 12 (as Example 2) | 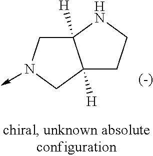 | ¹H NMR (400 MHz, MeOH- d₄) δ 8.57 (d, J = 2.40 Hz, 1H), 8.12 (d, J = 2.40 Hz, 1H), 7.92 (dd, J = 8.4, 2.80 Hz, 1H), 7.74 (dd, J = 8.4, 2.80 Hz, 1H), 7.58 (d, J = 8.40 Hz, 1H), 7.52 (d, J = 7.60 Hz, 1H), 6.86 (d, J = 9.20 Hz, 1H), 6.31 (dd, J = 7.6, 2.40 Hz, 1H), 6.10 (d, J = 2.80 Hz, 1H), 5.23 (s, 2H), 4.45 (m, 1H), 4.09 (m, 1H), 3.7-3.9 (m, 2H), 3.52-3.59 (m, 1H), 3.31-3.52 (m, 3H), 2.29-2.39 (m, 1H), 2.03-2.12 (m, 1H) | ES-LCMS m/z 424 |
| 13 (as Example 1) | 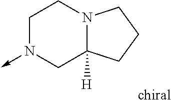 | ¹H NMR (400 MHz, MeOH- d₄) δ 8.58 (d, J = 2.0 Hz, 1H), 8.15 (s, 1H), 7.93 (dd, J = 8.4, 2.4 Hz, 1H), 7.52-7.66 (m, 3H), 7.0 (m, 1H), 6.32 (m, 1H), 6.08 (d, J = 2.4, 1H), 5.25 (s, 2H), 4.60-4.80 (m, 1H), 3.0-4.1 (m, 8H), 1.80-2.30 (m, 4H) | ES-LCMS m/z 438 |
| 14 (as Example 2) | 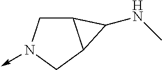 | ¹H NMR (400 MHz, MeOH- d₄) δ 8.59 (d, J = 2.4 Hz, 1H), 8.07 (d, J = 2.8 Hz, 1H), 7.94 (dd, J = 8.4, 2.4 Hz, 1H), 7.68 (dd, J = 8.8, 2.8 Hz, 1H), 7.60 (d, J = 8.4 Hz, 2H), 6.76 (d, J = 9.2 Hz, 1H), 6.33 (dd, J = 7.6, 2.8 Hz, 1H), 6.09 (d, J = 2.4 Hz, 1H), 5.25 (s, 2H), 3.92 (d, J = 10.4 Hz, 2H), 3.64 (d, J = 10.4 Hz, 2H), 2.84 (s, 3H), 2.68 (s, 1H), 2.32 (s, 2H) | ES-LCMS m/z 424 |
| 15 (as Example 2) | 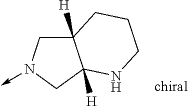 | ¹H NMR (400 MHz, MeOH- d₄) δ 8.56 (d, J = 2.4 Hz, 1H), 8.12 (d, J = 2.4 Hz, 1H), 7.90 (dd, J = 8.4, 2.4 Hz, 1H), 7.79 (dd, J = 9.2, 2.4 Hz, 1H), 7.51 (m, 2H), 6.89 (d, J = 9.6 Hz, 1H), 6.30 (dd, J = 7.6, 2.8 Hz, 1H), 6.06 (d, J = 2.8 Hz, 1H), 5.23 (s, 2H), 4.01 (d, J = 1.2 Hz, 1H), 3.89 (s, 2H), 3.74 (m, 1H), 3.57 (m, 1H), 3.37 (m, 1H), 3.05 (m, 1H), 2.95 (m, 1H), 1.95 (m, 2H), 1.84 (m, 2H) | ES-LCMS m/z 437 |
| 16 (as Example 2) | 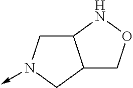 | ¹H NMR (400 MHz, MeOH- d₄) δ 8.52 (s, 1H), 8.15 (s, 1H), 7.63-7.80 (m, 2H), 7.37 (d, J = 8.4 Hz, 1H), 6.61 (s, 1H), 5.98-6.18 (m, 2H), 4.98- 5.40 (m, 5H), 4.40 (s, 1H), 3.31-4.00 (m, 6H) | ES-LCMS m/z 426 |
| 17 (as Example 2) | 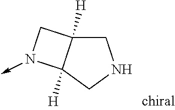 | ¹H NMR (400 MHz, MeOH- d₄) δ 8.56 (d, J = 2.4 Hz, 1H), 8.08 (d, J = 2.4 Hz, 1H), 7.91 (dd, J = 2.4, 8.4 Hz, 1H), 7.55-7.65 (m, 2H), 7.46 (d, J = 7.6 Hz, 1H), 6.61 (d, J = 8.8 Hz, 1H), 6.30 (dd, J = 2.8, 7.6 Hz, 1H), 6.06 (d, J = 2.8 Hz, 1H), 5.47 (s, 1H), 5.23 (s, 1H), 5.08-5.11 (m, 1H), 4.14-4.24 (m, 1H), 3.68- 3.86 (m, 3H), 3.40-3.51 (m, 1H), 3.30-3.40 (m, 1H), 3.19- 3.28 (m, 1H) | ES-LCMS m/z 410 |
| 18 (as Example 1) | 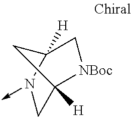 | ¹H NMR (400 MHz, MeOH- d₄) δ 8.57 (d, J = 2.0 Hz, 1H), 8.07 (d, J = 2.4 Hz, 1H), 7.87 (m, 2H), 7.51-7.53 (m, 2H), 7.10 (d, J = 9.2 Hz, 1H), 6.33 (dd, J =7.8, 2.8 Hz, 1H), 6.08 (d, J = 2.8 Hz, 1H), 5.23 (s, 2H), 5.01 (s, 1H), 4.75 (s, 1H), 3.72 (m, 1H), 3.45-3.61 (m, 2H), 3.42 (m, 1H), 2.12 (s, 2H), 1.42 (m, 9H) | ES-LCMS m/z 510 |
| 19 (as Example 2) | 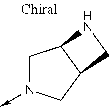 | ¹H NMR (400 MHz, MeOH- d₄) δ 8.51 (d, J = 2.0 Hz, 1H), 8.15 (d, J = 2.0 Hz, 1H), 7.82-7.94 (m, 2H), 7.52 (d, J = 8.0 Hz, 2H), 7.15 (d, J = 9.2 Hz, 1H), 6.27 (dd, J = 5.2, 7.6 Hz, 1H), 6.02 (d, J = 2.4 Hz, 1H), 5.17 (s, 2H), 5.07 (t, J = 6.8 Hz, 1H), 4.41 (d, J = 13.2 Hz, 1H), 4.21 (t, J = 11.2 Hz, 1H), 4.11 (d, J = 11.2 Hz, 1H), 3.68-3.76 (m, 1H), 3.50-3.60 (m, 2H), 3.43- 3.50 (m, 1H) | ES-LCMS m/z 410 |
| 20 (as Example 1) | 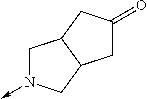 | ¹H NMR (400 MHz, MeOH- d₄) δ 8.56 (d, J = 2.40 Hz, 1H), 8.05 (d, J = 2.40 Hz, 1H), 7.90 (dd, J = 8.4, 2.4 Hz, 1H), 7.85-7.70 (m, 1H), 7.58-7.51 (m, 2H), 6.90 (d, J = 9.6 Hz, 1H), 6.30 (dd, J = 7.6, 2.4 Hz, 1H), 6.06 (d, J = 2.80 Hz, 1H), 5.23 (s, 2H), 3.96-3.80 (m, 2H), 3.49-3.45 (m, 2H), 3.30-3.27 (m, 2H), 2.27-2.26 (m, 2H), 2.31-2.19 (m, 2H) | ES-LCMS m/z 437 |
| 21 (as Example 1) | 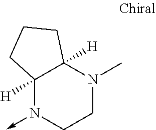 | ¹H NMR (400 MHz, CDCl₃) δ 8.60 (s, 1H), 8.16 (s, 1H), 7.95 (d, J = 8.0 Hz, 1H), 7.66-7.54 (m, 3H), 7.02 (d, J = 8.8 Hz, 1H), 6.34 (d, J = 7.6 Hz, 1H), 6.10 (s, 1H), 5.26 (s, 2H), 4.88 (m, 1H), 4.57 (s, 1H), 3.66-3.56 (m, 2H), 3.28-3.253 (m, 2H), 2.99 (s, 3H), 2.28 (m, 1H), 2.11-1.86 (m, 5H) | ES-LCMS m/z 452 |
| 22 (as Example 1) | 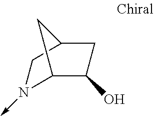 | ¹H NMR (400 MHz, MeOH- d₄) δ 8.78 (d, J = 0.4 Hz, 1H), 8.23 (dd, J = 2.4 Hz, 8.4 Hz, 1H), 8.04 (d, J = 2.4 Hz, 1H), 7.94 (dd, J = 2.4 Hz, 10 Hz, 1H), 7.81 (d, J = 8.8 Hz, 1H), 7.62 (d, J = 7.6 Hz, 1H), 7.00-7.40 (m, 1H), 6.40 (dd, J = 2.8 Hz, 7.6 Hz, 1H), 6.14 (d, J = 2.4 Hz, 1H), 5.37 (s, 2H), 6.28 (dd, J = 2.8 Hz, 7.6 Hz, 1H), 6.03 (d, J = 2.8 Hz, 1H), 5.22 (s, 2H), 4.68 (s, 1H), 4.06 (d, J = 6.8 Hz, 1H), 3.56-3.64 (m, 1H), 3.10-3.20 (m, 1H), 2.72 (s, 1H), 2.08- 2.18 (m, 2H), 1.81-1.83 (m, 1H), 1.63-1.65 (m, 1H) | ES-LCMS m/z 425 |
| 23 (as Example 1) | 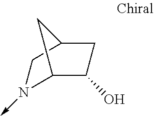 | ¹H NMR (400 MHz, MeOH- d₄) δ 8.78 (d, J = 0.4 Hz, 1H), 8.23 (dd, J = 2.4 Hz, 8.4 Hz, 1H), 8.04 (d, J = 2.4 Hz, 1H), 7.94 (dd, J = 2.4 Hz, 10 Hz, 1H), 7.81 (d, J = 8.8 Hz, 1H), 7.62 (d, J = 7.6 Hz, 1H), 7.00-7.40 (m, 1H), 6.40 (dd, J = 2.8 Hz, 7.6 Hz, 1H), 6.14 (d, J = 2.4 Hz, 1H), 5.37 (s, 2H), 6.28 (dd, J = 2.8 Hz, 7.6 Hz, 1H), 6.03 (d, J = 2.8 Hz, 1H), 5.22 (s, 2H), 4.68 (s, 1H), 4.06 (d, J = 6.8 Hz, 1H), 3.56-3.64 (m, 1H), 3.10-3.20 (m, 1H), 2.72 (s, 1H), 2.08- 2.18 (m, 2H), 1.81-1.83 (m, 1H), 1.63-1.65 (m, 1H) | ES-LCMS m/z 425 |
| 24 (as Example 1) | 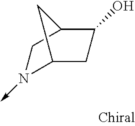 | ¹H NMR (400 MHz, MeOH- d₄) δ 8.78 (d, J = 0.4 Hz, 1H), 8.23 (dd, J = 2.4 Hz, 8.4 Hz, 1H), 8.04 (d, J = 2.4 Hz, 1H), 7.94 (dd, J = 2.4 Hz, 10 Hz, 1H), 7.81 (d, J = 8.8 Hz, 1H), 7.62 (d, J = 7.6 Hz, 1H), 7.00-7.40 (m, 1H), 6.40 (dd, J = 2.8 Hz, 7.6 Hz, 1H), 6.14 (d, J = 2.4 Hz, 1H), 5.37 (s, 2H), 6.28 (dd, J = 2.8 Hz, 7.6 Hz, 1H), 6.03 (d, J = 2.8 Hz, 1H), 5.22 (s, 2H), 4.68 (s, 1H), 4.06 (d, J = 6.8 Hz, 1H), 3.56-3.64 (m, 1H), 3.10-3.20 (m, 1H), 2.72 (s, 1H), 2.08- 2.18 (m, 2H), 1.81-1.83 (m, 1H), 1.63-1.65 (m, 1H) | ES-LCMS m/z 425 |
| 25 (as Example 1) | 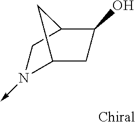 | ¹H NMR (400 MHz, MeOH- d₄) δ 8.78 (d, J = 0.4 Hz, 1H), 8.23 (dd, J = 2.4 Hz, 8.4 Hz, 1H), 8.04 (d, J = 2.4 Hz, 1H), 7.94 (dd, J = 2.4 Hz, 10 Hz, 1H), 7.81 (d, J = 8.8 Hz, 1H), 7.62 (d, J = 7.6 Hz, 1H), 7.00-7.40 (m, 1H), 6.40 (dd, J = 2.8 Hz, 7.6 Hz, 1H), 6.14 (d, J = 2.4 Hz, 1H), 5.37 (s, 2H), 6.28 (dd, J = 2.8 Hz, 7.6 Hz, 1H), 6.03 (d, J = 2.8 Hz, 1H), 5.22 (s, 2H), 4.68 (s, 1H), 4.06 (d, J = 6.8 Hz, 1H), 3.56-3.64 (m, 1H), 3.10-3.20 (m, 1H), 2.72 (s, 1H), 2.08- 2.18 (m, 2H), 1.81-1.83 (m, 1H), 1.63-1.65 (m, 1H) | ES-LCMS m/z 425 |
| 26 (as Example 2) | 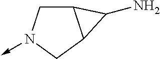 | ¹H NMR (400 MHz, MeOH- d₄) δ 8.74 (d, J = 0.8 Hz, 1H), 8.15-8.17 (m, 2H), 8.08- 8.10 (m, 1H), 7.77 (d, J = 8.8 Hz, 1H), 7.64 (d, J = 7.6 Hz, 1H), 7.20 (d, J = 10 Hz, 1H), 6.40 (dd, J = 2.8, 7.6 Hz, 1H), 6.13 (d, J = 8.0 Hz, 3H), 5.35 (s, 2H), 3.99-4.03 (m, 2H), 3.90-3.98 (m, 2H), 2.67 (s, 1H), 2.42 (s, 2H) | ES-LCMS m/z 410 |
| 27 (as Example 2) | 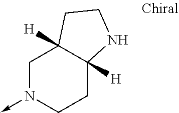 | ¹H NMR (400 MHz, MeOH- d₄) δ 8.58 (m, 1H), 8.08 (d, J = 2.00 Hz, 1H), 7.92 (dd, J = 8.40, 2.40 Hz, 1H), 7.55 (m, 3H), 6.91 (d, J = 9.20 Hz, 1H), 6.30 (dd, J =7.60, 2.40 Hz, 1H), 6.07 (d, J = 2.80 Hz, 1H), 5.24 (s, 2H), 4.85 (m, 1H), 4.01 (m, 1H), 3.40 (m, 1H), 3.22 (m, 1H), 3.01 (m, 1H), 2.25 (m, 1H), 1.84 (m, 3H), 1.63 (m, 2H), 1.36 (m, 1H) | ES-LCMS m/z 438 |
| 28 (as Example 2) | 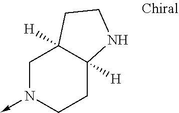 | ¹H NMR (400 MHz, MeOH- d₄) δ 8.58 (d, J = 2.40 Hz, 1H), 8.08 (d, J = 2.80 Hz, 1H), 7.92 (dd, J = 8.40, 2.40 Hz, 1H), 7.55 (m, 3H), 6.91 (d, J = 9.20 Hz, 1H), 6.30 (dd, J = 7.60, 2.80 Hz, 1H), 6.07 (d, J = 2.80 Hz, 1H), 5.24 (s, 2H), 4.94 (m, 1H), 4.01 (m, 1H), 3.42 (m, 1H), 3.22 (m, 1H), 3.01 (m, 1H), 2.21 (m, 1H), 1.83 (m, 3H), 1.51 (m, 2H), 1.31 (m, 1H) | ES-LCMS m/z 438 |
| 29 (as Example 2) | 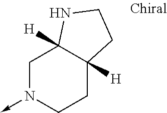 | ¹H NMR (400 MHz, MeOH- d₄) δ 8.58 (d, J = 2.40 Hz, 1H), 8.07 (d, J = 2.80 Hz, 1H), 7.92 (dd, J = 8.40, 2.40 Hz, 1H), 7.55 (m, 3H), 6.91 (d, J = 9.20 Hz, 1H), 6.30 (dd, J = 7.60, 2.80 Hz, 1H), 6.07 (d, J = 2.80 Hz, 1H), 5.24 (s, 2H), 4.24 (d, 1H), 3.92 (m, 1H), 3.62 (m, 1H), 3.53 (m, 1H), 3.21 (m, 1H), 3.16 (m, 1H), 2.53 (m, 1H), 2.10 (m, 1H), 1.92 (m, 1H), 1.83 (m, 1H), 1.65 (m, 1H), 1.25 (m, 1H) | ES-LCMS m/z 438 |
| 30 (as Example 2) | 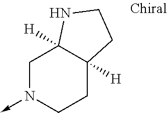 | ¹H NMR (400 MHz, MeOH- d₄) δ 8.58 (d, J = 2.00 Hz, 1H), 8.07 (d, J = 2.40 Hz, 1H), 7.92 (dd, J = 8.40, 2.40 Hz, 1H), 7.55 (m, 3H), 6.91 (d, J = 9.20 Hz, 1H), 6.30 (dd, J = 7.60, 2.80 Hz, 1H), 6.07 (d, J = 2.40 Hz, 1H), 5.24 (s, 2H), 4.24 (d, J = 4.80 Hz, 1H), 3.92 (m, 1H), 3.62 (m, 2H), 3.20 (m, 2H), 2.51 (m, 1H), 2.18 (m, 1H), 1.92 (m, 1H), 1.81 (m, 1H), 1.63 (m, 1H), 1.25 (m, 1H) | ES-LCMS m/z 438 |
| 31 (as Example 2) | 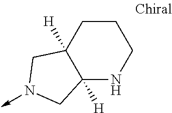 | ¹H NMR (400 MHz, MeOH- d₄) δ 8.60 (d, J = 2.0 Hz, 1H), 8.21 (d, J = 2.4 Hz, 1H), 8.11 (d, J = 2.4 Hz, 1H), 8.08 (d, J = 2.4 Hz, 1H), 7.95 (dd, J = 8.4 Hz, 2.4 Hz, 2H), 7.61 (d, J = 7.6 Hz, 1H), 6.36 (dd, J = 7.6 Hz, 2.4 Hz, 1H), 6.10 (d, J = 2.8 Hz, 1H), 5.26 (s, 2H), 4.06-4.13 (m, 2H), 3.88- 3.95 (m, 2H), 3.73 (m, 1H), 3.43 (m, 1H), 3.01-3.11 (m, 2H), 1.97-1.98 (m, 4H) | ES-LCMS m/z 438 |
| 32 (as Example 2) | 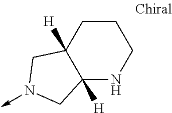 | ¹H NMR (400 MHz, MeOH- d₄) δ 8.66 (d, J = 2.0 Hz, 1H), 8.21 (d, J = 2.4 Hz, 1H), 8.11 (dd, J = 9.6 Hz, 2.4 Hz, 1H), 8.05 (dd, J = 8.4 Hz, 2.4 Hz, 1H), 7.69 (d, J = 8.4 Hz, 1H), 7.66 (d, J = 7.2 Hz, 1H), 7.26 (d, J = 9.2 Hz, 1H), 6.38 (dd, J = 7.6 Hz, 2.4 Hz, 1H), 6.11 (d, J = 2.8 Hz, 1H), 5.30 (s, 2H), 4.08-4.15 (m, 2H), 3.90-3.99 (m, 2H), 3.80-3.83 (m, 1H), 3.42 (m, 1H), 3.01- 3.11 (m, 2H), 1.87-1.98 (m, 4H) | ES-LCMS m/z 438 |

MCHR¹ pIC₅₀ Determination FLIPR™ Assay: HEK293 cells stably transfected with hMCHR1 were propagated as adherent cultures at 37° C. in a humidified incubator. Cells were split 1:8 at 90% confluency two times per week. New cell stocks were recovered from storage every two months. Cells were plated in black 384-well plates (Greiner) 24 hours prior to assay at 15,000 cells/well in 50 μL DMEM/F12, 10% FBS, 2 mM I-glutamine. Compounds to be profiled were prepared by making a stock solution at 3×10⁻³M in 100% DMSO. The stock solutions were serially diluted 1:4 in 100% DMSO using JANUS (PerkinElmer) liquid handling instrument to allow for an 11 point curve in singlicate. At the time of the assay, the media was removed from the cell plate by aspiration, followed by the addition of 20 μL of loading buffer (Calcium 4 Kit, Molecular Dynamics corporation). Following 50 min incubation at 37° C., 10 μL of compound was added to the plates via the FLIPR™ instrument (Molecular Dynamics corporation). The plates were incubated at room temperature for 15 minutes along with an MCH peptide agonist challenge plate. On the FLIPR™, a basal response was collected over 10 seconds followed by the addition of 10 μL of MCH challenge concentration at 4XEC₅₀. Data was collected over 4 minutes and subjected to a nonlinear regression analysis curve fitting program to generate pIC₅₀s.
MCHR1 pIC₅₀ Determination Reporter Gene Assay: The assay consists of cells plated at ten thousand cells/well in DMEM/F12, 5% FBS, 2 mM I-glutamine in black 384-well assay plates. The day after plating, the media was removed by aspiration seventeen hours prior to assay, followed by the addition of 50 μL of media without serum to reduce background signal noise. Compounds were prepared by making a stock solution at 3×10⁻3M. The stock solutions is serially diluted 1:4 in 100% DMSO using the JANUS liquid handling instrument (Perkin Elmer) to allow for an 11 point curves in singlicate. On the day of the assay, compounds (0.5 μL) were pipetted into the assay plate using JANUS. Following incubation for 45 minutes at 37° C., 10 μL of 6×EC₈₀ concentration (6×50 nM) of MCH was added to the plate allowing for appropriate controls. The plates were then incubated under the same conditions for five hours. Under subdued light conditions, the compound/assay solution was removed by aspiration from the plates, followed by the addition of 15 μL per well SteadyGlo™ reagent using a Multidrop. Plates were then sealed with self-adhesive clear plate seals and wiped with a static free dryer sheet to reduce false counts due to static charge and placed on the shaker for 8 min in dark. The amount of luciferase generated was quantified in a TopCount (PerkinElmer Packard) at 19.8° C. in SPC (single photon counting) mode with a 5 second count/well and subjected to a nonlinear regression analysis curve fitting program to generate pIC₅₀s.

Although specific embodiments of the present invention are herein illustrated and described in detail, the invention is not limited thereto. The above-detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

Exemplified compounds of the present invention were tested according to the above assays and were found to be functional antagonists of MCH at MCHR1. The IC₅₀s in the FLIPR™ assay ranged from about 10 nM to 0.5 uM. The majority of the compounds were under 60 nM; the most active compounds were ≦50 nM.

The compound of Example 5 was tested generally according to the assays described herein and in at least one experimental run exhibited an IC₅₀ value equal to 29 nM in the FLIPR™ assay.

The compound of Example 14 was tested generally according to the assays described herein and in at least one experimental run exhibited an IC₅₀ value equal to 45 nM in the FLIPR™ assay.

The compound of Example 16 was tested generally according to the assays described herein and in at least one experimental run exhibited an IC₅₀ value equal to 56 nM in the FLIPR™ assay.

What is claimed is:

1. A compound of Formula (I), 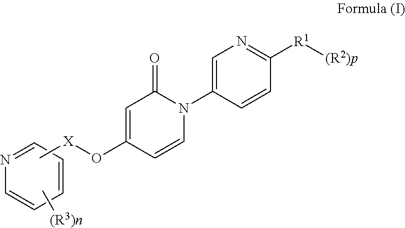
or a pharmaceutically acceptable salt thereof wherein:
R¹ is a saturated 6-10 membered heterocyclic ring system containing 1 or 2 ring nitrogen atoms and an optional ring oxygen atom, which ring system incorporates fused or bridged rings and which is attached to the pyridine ring via a nitrogen atom;
R² is selected from the group consisting of: hydrogen, C₁₋₆alkyl, hydroxyl, —C(O)NR^{a}R^{b}, —C(O)R^{a}, —SO₂R^{a}, —C(O)OR^{a}, oxo, —C(O)NR^{a}R^{b}, —NR^{a}R^{b}, —NR^{a}C(O)R^{b}, —NR^{a}C(O)OR^{b}, and —NR^{a}SO₂R^{b};
R^{a} is selected from the group consisting of: hydrogen, C₁₋₆alkyl, or C₃₋₆cycloalkyl;
R^{b} is selected from the group consisting of: hydrogen, C₁₋₆alkyl, C₃₋₆cycloalkyl, aryl, and heteroaryl;
or R^{a} and R^{b} together with the nitrogen to which they are attached form a heterocycloalkyl, and said heterocycloalkyl is optionally substituted with one, two, or three R^{c} groups;
R³ is H, F, Cl, C₁₋₃alkyl, Cyclopropyl, C₁₋₃alkoxy, amino, C₁₋₃alkylamino, oxo, or CN;
R^{c} is H, F, Cl, C₁₋₆alkyl, C₃₋₆cycloalkyl, C₁₋₃alkoxy, amino, C₁₋₃alkylamino, oxo, and CN;
X is (CH₂)ₘ;
m is 0-2;
n is 0-3;
p is 0-3;
with the proviso that R² is not —NR^{a}COOH or —NR^{a}SO₂H.

2. The compound of claim 1, which is represented by Formula (I)(A) 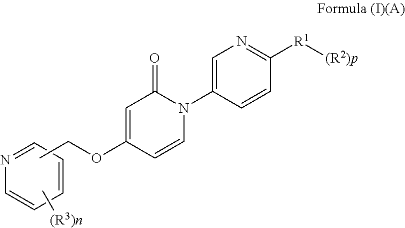
or a pharmaceutically acceptable salt thereof wherein:
R1 is a saturated 6-12 membered heterocyclic ring system containing 1 or 2 ring nitrogen atoms and an optional ring oxygen atom, which ring system incorporates fused or bridged rings and which is attached to the pyridine ring via a nitrogen atom;
R2 is selected from the group consisting of: hydrogen, C₁₋₆alkyl, hydroxyl, —C(O)NR^{a}R^{b}, —C(O)R^{a}, —SO₂R^{a}, —C(O)OR^{a}, oxo, —C(O)NR^{a}R^{b}, —NR^{a}R^{b}, —NR^{a}C(O)R^{b}, —NR^{a}C(O)OR^{b}, and —NR^{a}SO₂R^{b};
R^{a} is selected from the group consisting of: hydrogen, C₁₋₆alkyl, and C₃₋₆cycloalkyl;
R^{b} is selected from the group consisting of: hydrogen, C₁₋₆alkyl, and C₃₋₆cycloalkyl;
or R^{a} and R^{b} together with the nitrogen to which they are attached form a heterocycloalkyl, and said heterocycloalkyl is optionally substituted with one, two, or three R^{c} groups;
R³ is H, F, Cl, C₁₋₃alkyl, cyclopropyl, C₁₋₃alkoxy, amino, C₁₋₃alkylamino, oxo, or CN;
R^{c} is H, F, Cl, C₁₋₆alkyl, C₃₋₆cycloalkyl, C₁₋₃alkoxy, amino, C₁₋₃alkylamino, oxo, or CN;
;
n is 0-3;
p is 0-3;
with the proviso that R² is not —NR^{a}COOH or —NR^{a}SO₂H.

3. The compound of claim 1, which is represented by Formula (I)(B) 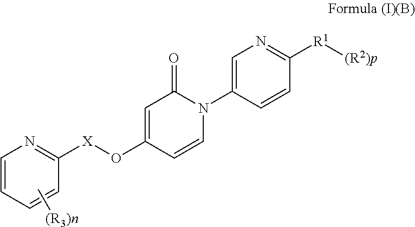
or a pharmaceutically acceptable salt thereof wherein:
R1 is a saturated 6-12 membered heterocyclic ring system containing 1 or 2 ring nitrogen atoms and an optional ring oxygen atom, which ring system incorporates fused or bridged rings and which is attached to the pyridine ring via a nitrogen atom;
R2 is selected from the group consisting of: hydrogen, C₁₋₆alkyl, hydroxyl, —C(O)NR^{a}R^{b}, —C(O)R^{a}, —SO₂R^{a}, —C(O)OR^{a}, oxo, —C(O)NR^{a}R^{b}, —NR^{a}R^{b}, —NR^{a}C(O)R^{b}, —NR^{a}C(O)OR^{b}, and —NR^{a}SO₂R^{b};
R^{a} is selected from the group consisting of: hydrogen, C₁₋₆alkyl, and C₃₋₆cycloalkyl;
R^{b} is selected from the group consisting of: hydrogen, C₁₋₆alkyl, and C₃₋₆cycloalkyl;
or R^{a} and R^{b} together with the nitrogen to which they are attached form a heterocycloalkyl, and said heterocycloalkyl is optionally substituted with one, two, or three R^{c} groups;
R³ is H, F, Cl, C₁₋₃alkyl, cyclopropyl, C₁₋₃alkoxy, amino, C₁₋₃alkylamino, oxo, or CN;
R^{c} is H, F, Cl, C₁₋₆alkyl, C₃₋₆cycloalkyl, C₁₋₃alkoxy, amino, C₁₋₃alkylamino, oxo, or CN;
n is 0-3;
p is 0-3;
with the proviso that R² is not —NR^{a}COOH or —NR^{a}SO₂H.

4. The compound of claim 1, which is represented by Formula (I)(C) 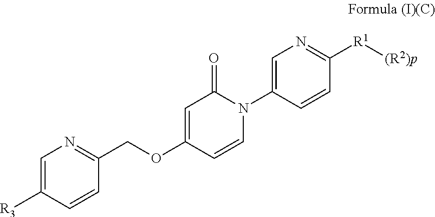
or a pharmaceutically acceptable salt thereof wherein:
R1 is a saturated 6-12 membered heterocyclic ring system containing 1 or 2 ring nitrogen atoms and an optional ring oxygen atom, which ring system incorporates fused or bridged rings and which is attached to the pyridine ring via a nitrogen atom
R2 is selected from the group consisting of: hydrogen, C₁₋₆alkyl, hydroxyl, —C(O)NR^{a}R^{b}, —C(O)R^{a}, —SO₂R^{a}, —C(O)OR^{a}, oxo, —C(O)NR^{a}R^{b}, —NR^{a}R^{b}, —NR^{a}C(O)R^{b}, —NR^{a}C(O)OR^{b}, and —NR^{a}SO₂R^{b};
R^{a} is selected from the group consisting of: hydrogen, C₁₋₆alkyl, and C₃₋₆cycloalkyl;
R^{b} is selected from the group consisting of: hydrogen, C₁₋₆alkyl, and C₃₋₆cycloalkyl;
or R^{a} and R^{b} together with the nitrogen to which they are attached form a heterocycloalkyl, and said heterocycloalkyl is optionally substituted with one, two, or three R^{c} groups;
R³ is H, F, Cl, C₁₋₃alkyl, cyclopropyl, C₁₋₃alkoxy, amino, C₁₋₃alkylamino, oxo, or CN;
R^{c} is H, F, Cl, C₁₋₆alkyl, C₃₋₆cycloalkyl, C₁₋₃alkoxy, amino, C₁₋₃alkylamino, oxo, or CN;
p is 0-3;
with the proviso that R² is not —NR^{a}COOH or —NR^{a}SO₂H.

5. The compound according to claim 1, R1 is a bicyclic or bridged ring system selected from: octahydropyrrolopyrrole, octahydropyrrolopyridine, 3,6-diazabicyclo[3.2.0]hept-6-yl, 6-amino-3-azabicyclo[3.1.0]hex-3-yl, and azabicyclo[3.1.0]hexan-1-yl.

6. The compound of claim 1 or a pharmaceutically acceptable salt thereof, wherein R³ is F or Cl, R^{b} is selected from the group consisting of: hydrogen, C₁₋₆alkyl, and C₃₋₆cycloalkyl; m is 1; and n is 1.

7. The compound of claim 1 or a pharmaceutically acceptable salt thereof, wherein R^{a} is H or C₁₋₆alkyl; and R² is H or NR^{a}R^{b}.

8. The compound of claim 1 or a pharmaceutically acceptable salt thereof, wherein R² is H or a substituted C₁₋₆alkyl.

9. The compound of claim 1 or a pharmaceutically acceptable salt thereof wherein R^{a} and R^{b} are joined together with the nitrogen to which they are attached to form a heterocycle.

10. The compound of claim 1 or a pharmaceutically acceptable salt thereof wherein said heterocycle is substituted with one to three R^{c} groups.

11. The compound of claim 10 or a pharmaceutically acceptable salt thereof wherein said R^{c} is selected from the group consisting of substituted C₁₋₃alkoxy, substituted C₁₋₆alkyl, and substituted C₃₋₆cycloalkyl.

12. The compound of claim 1 or a pharmaceutically acceptable salt thereof, wherein m is 1.

13. The compound of claim 1 or a pharmaceutically acceptable salt thereof, wherein n is 0, 1, or 2.

14. The compound of claim 1 or a pharmaceutically acceptable salt thereof, wherein p is 1 or 2.

15. The compound of claim 1.

16. A pharmaceutical composition comprising a compound of claim 1 or salt thereof and one or more excipients.

17. A method of treatment comprising the administering to a human in need thereof a pharmaceutical composition comprising a compound of claim 1 or a pharmaceutically acceptable salt thereof and at least one excipient, wherein said treatment is for obesity, diabetes, hypertension, depression, anxiety, drug addiction, substance addiction, or a combination thereof.

18. The method of claim 17 wherein said treatment is for obesity, diabetes, or both.

19-22. (canceled)
